SEQUENCE LISTING (21 sequences)

SEQ ID NO: 1 - protein, 836 aa, Artificial Sequence
  Description of Artificial Sequence Synthetic polypeptide
RTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 2 - DNA, 1502 bp, Artificial Sequence
  Description of Artificial Sequence Synthetic polynucleotide
agagacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 2217 bp, Artificial Sequence
  Description of Artificial Sequence Synthetic polynucleotide
gctgtgtggcccaggcttttctcaaactcctgagggcaagcgatcctcccacctcagcctcctgagtagctgggactacaggcatgtgccactagacctggctctaaagacatatatgacacacgaaaccatttatttttcatttcacaatgtttattcacatatatggtattagtattctaatgtagtgatgcactctaaatttgcattatatttcctagaacatctgaacagagcataggaaattccctattttgccattatcagttctaacaaaaatcttaaaagcactttatcatttcatttccctgcactgtaatttttttaaatgatcaaaaacagtatcataccaaggcttacttatattggaatactattttagaaagttgtgggctgggttgtatttataaatcttgttggtcagatgtctgcaatgagtaaatttagcaccattatcaggaagctttctcaccaatgacaacttcattggaagattttaatgaaagtgtagcatactctagggaaaaaatatgaatattttagcatctatgtattgaaaattatgttgaataaatgtcagactattttttacataacgttgcttctgtttaattttgtcacgttcagaggtggggggtaggagatgtaagcccttgacagcaaaataattccttttgcttgatttcagacagttgcatcagctcctttgttctgtgttcatgttacacttatttaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgacaaaaaaaaaaaaaaaaaa

SEQ ID NO: 4 - DNA, 1684 bp, Artificial Sequence
  Description of Artificial Sequence Synthetic polynucleotide
ggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 5 - DNA, 1878 bp, Artificial Sequence
  Description of Artificial Sequence Synthetic polynucleotide
ccacgcgtccgggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaa

SEQ ID NO: 6 - DNA, 1864 bp, Artificial Sequence
  Description of Artificial Sequence Synthetic polynucleotide
aacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaaaaaaaaaaaaaaaaaa

SEQ ID NO: 7 - DNA, 1871 bp, Artificial Sequence
  Description of Artificial Sequence Synthetic polynucleotide
gaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaaaaaaaaaaaaaaaaaaaa

SEQ ID NO: 8 - DNA, 1865 bp, Artificial Sequence
  Description of Artificial Sequence Synthetic polynucleotide
agagacgtgaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 9 - DNA, 1884 bp, Artificial Sequence
  Description of Artificial Sequence Synthetic polynucleotide
gacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 10 - DNA, 1860 bp, Artificial Sequence
  Description of Artificial Sequence Synthetic polynucleotide
cgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtgtttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtaagatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 11 - DNA, 1884 bp, Artificial Sequence; identical to SEQ ID NO: 9
  Description of Artificial Sequence Synthetic polynucleotide

SEQ ID NO: 12 - protein, 232 aa, Artificial Sequence
  Description of Artificial Sequence Synthetic polypeptide
MESETEPEPVTLLVKSPNQRHRDLELSGDRGWSVGHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 13 - protein, 209 aa, Artificial Sequence
  Description of Artificial Sequence Synthetic polypeptide
MQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 14 - protein, 356 aa, Artificial Sequence
  Description of Artificial Sequence Synthetic polypeptide
GHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 15 - protein, 391 aa, Artificial Sequence
  Description of Artificial Sequence Synthetic polypeptide
MESETEPEPVTLLVKSPNQRHRDLELSGDRGWSVGHLKAHLSRVYPERPRPEDQRLIYSGKLLLDHQCLRDLLPKQEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 16 - DNA, 1857 bp, Artificial Sequence
  Description of Artificial Sequence Synthetic polynucleotide
aagacaccaagtgtcgttgtggggtcgcagacggctgcgtcgccgcccgttcggcatccctgagcgcagtcgagcctccagcgccgcagacatggagcccgagccacagcccgagccggtcacgctgctggtgaagagccccaatcagcgccaccgcgacttggagctgagtggcgaccgcggttggagtgtgagtcgcctcaaggcccacctgagccgagtctaccccgaacgcccgcgcccagaggaccagaggttaatttattctgggaagctgctgttggatcaccaatgtctccaagacttgcttccaaagcaggaaaagcgacatgttttgcacctcgtgtgcaatgtgaggagtccctcaaaaaagccagaagccagcacaaagggtgctgagtccacagagcagccggacaacactagtcaggcacagtatcctggggattcctcaagcgatggcttacgggaaagggaagtccttcggaaccttcctccctctggatgggagaacgtctctaggcctgaagccgtccagcagactttccaaggcctcgggcccggcttctctggctacaccacctacgggtggctgcagctctcctggttccagcagatctatgcaagacagtactacatgcaatacttggctgccactgctgcttcaggagcttttggccctacaccaagtgcacaagaaatacctgtggtctctacaccggctcccgcccctatacacaaccagtttccggcagaaaaccagccggccaatcagaatgcagccgctcaagcggttgttaatcccggagccaatcagaacttgcggatgaatgcacaaggcggccctctggtggaagaagatgatgagataaaccgagactggttggattggacctactcagcagcgacattttccgttttcctcagcattctttacttctactcctccctgagcagattcctcatggtcatgggcgccaccgtagtcatgtacctgcaccacgtcgggtggtttccattcagacagaggccagttcagaacttcccagatgacggtccccctcaggaagctgccaaccaggaccccaacaataacctccagggaggtttggaccctgaaatggaagaccccaaccgcctccccgtaggccgtgaagtgctggaccctgagcataccagcccctcgttcatgagcacagcatggctagtcttcaagactttctttgcctctcttcttccggaaggcccaccagccctagcaaactgatggcccctgtgctctgttgctggaggctttcacagcttggactggatcgtcccctggcgtggactcgagagagtcattgaaaacccacaggatgacgatgtgcttctgtgccaagcaaaagcacaaactaagacatgaagccgtggtacaaactgaacagggcccctcatgtcgttattctgaagagctttaatgtatactgtatgtagtctcataggcactgtaaacagaaggcccagggtcgcatgttctgcctgagcacctccccagacgtgtgtgcatgtgtgccgtacatggaagtcatagacgtgtgtgcatgtgtgctctacatggaagtcatagatgcagaaacggttctgctggttcgatttgattcctgttggaatgttgcaattacactaagtgtactactttatataatcagtgacttgctagacatgttagcaggacttttctaggagagacttattgtatcattgctttttaaaacgcagtgcttacttactgagggcggcgacttggcacaggtaaagcctttgccgggttttctgttcaataaagttttgctatgaacgacaaaaaaaaaaaaa

SEQ ID NO: 17 - protein, 391 aa, Artificial Sequence
  Description of Artificial Sequence Synthetic polypeptide
MEPEPQPEPVTLLVKSPNQRHRDLELSGDRGWSVSRLKAHLSRVYPERPRPEDQRLIYSGKLLLDHQCLQDLLPKQEKRHVLHLVCNVRSPSKKPEASTKGAESTEQPDNTSQAQYPGDSSSDGLREREVLRNLPPSGWENVSRPEAVQQTFQGLGPGFSGYTTYGWLQLSWFQQIYARQYYMQYLAATAASGAFGPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGPPQEAANQDPNNNLQGGLDPEMEDPNRLPVGREVLDPEHTSPSFMSTAWLVFKTFFASLLPEGPPALAN

SEQ ID NO: 18 - DNA, 1871 bp, Artificial Sequence
  Description of Artificial Sequence Synthetic polynucleotide
aaagacgccaagtgtcgttgtgtggtctcagacggctgcgtcgccgcccgttcggcatccctgagcgcagtcgagccgccagcgacgcagacatggagcccgagccacagcccgagccggtcacgctgctggtgaagagtcccaatcagcgccaccgcgacttggagctgagtggcgaccgcagttggagtgtgagtcgcctcaaggcccacctgagccgagtctaccccgagcgcccgcgtccagaggaccagaggttaatttattctgggaagctgctgttggatcaccagtgtctccaagatttgcttccaaagcaggaaaagcgacatgttttgcaccttgtgtgcaatgtgaagaatccctccaaaatgccagaaaccagcacaaagggtgctgaatccacagagcagccggacaactctaatcagacacagcatcctggggactcctcaagtgatggtttacggcaaagagaagttcttcggaacctttctccctccggatgggagaacatctctaggcctgaggctgtccagcagactttccaaggcctggggcctggcttctctggctacacaacgtatgggtggctgcagctctcctggttccagcagatctatgcaaggcagtactacatgcaatacttagctgccactgctgcatcaggaacttttgtcccgacaccaagtgcacaagagatacctgtggtctctacacctgctccggctcctatacacaaccagtttccggcagaaaaccagccggccaatcagaatgcagctgctcaagcggttgtcaatcccggagccaatcagaacttgcggatgaatgcacaaggtggccccctggtggaggaagatgatgagataaaccgagactggttggattggacctattccgcagcgacgttttctgttttcctcagcatcctttacttctactcctcgctgagcagatttctcatggtcatgggtgccactgtagtcatgtacctgcaccacgtcgggtggtttccgttcagacagaggccagttcagaacttcccggatgatggtggtcctcgagatgctgccaaccaggaccccaacaataacctccagggaggtatggacccagaaatggaagaccccaaccgcctccccccagaccgcgaagtgctggaccctgagcacaccagcccctcgtttatgagcacagcatggctagtcttcaagactttctttgcctctcttcttccagaaggcccaccagccctagccaactgatggcccttgtgctctgtcgctggtggctttgacagctcggactggatcgtctggctccggctccttttcctcccctggcgtggactcgacagagtcattgaaaacccacaggatgacatgtgcttctgtgccaagcaaaagcacaaactaagacatgaagccgtggtacaaactgaacagggcccctcatgtcgttattctgaagagctttaatgtatactgtatgtagtttcataggcactgtaagcagaaggcccagggtcgcatgttctgcctgagcacctccccagatgtgtgtgcatgtgtgctgtacatggaagtcatagacgtgtgtgcatgtgtgctctacatggaagtcatagatgcagaaacggttctgctggttcgatttgattcctgttggaatgttcaaattacactaagtgtactactttatataatcagtgaattgctagacatgttagcaggacttttctaggagagacttatgtataattgctttttaaaatgcagtgctttcctttaaaccgagggtggcgacttggcagaggtaaaacctttgccgagttttctgttcaataaagttttgctatgaatgactgt

SEQ ID NO: 19 - protein, 391 aa, Artificial Sequence
  Description of Artificial Sequence Synthetic polypeptide
MEPEPQPEPVTLLVKSPNQRHRDLELSGDRSWSVSRLKAHLSRVYPERPRPEDQRLIYSGKLLLDHQCLQDLLPKQEKRHVLHLVCNVKNPSKMPETSTKGAESTEQPDNSNQTQHPGDSSSDGLRQREVLRNLSPSGWENISRPEAVQQTFQGLGPGFSGYTTYGWLQLSWFQQIYARQYYMQYLAATAASGTFVPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGGPRDAANQDPNNNLQGGMDPEMEDPNRLPPDREVLDPEHTSPSFMSTAWLVFKTFFASLLPEGPPALAN

SEQ ID NO: 20 - DNA, 21 bp, Artificial Sequence
  Description of Artificial Sequence Synthetic oligonucleotide
gggaaguucuucggaaccutt

SEQ ID NO: 21 - DNA, 21 bp, Artificial Sequence
  Description of Artificial Sequence Synthetic oligonucleotide
agguuccgaagaacuuccctt

(19) United States
(12) Patent Application Publication
(10) Pub. No.: US 2012/0015967 A1
(43) Pub. Date: Jan. 19, 2012

(54) PYRIMIDINE AND PYRIDINE DERIVATIVES AS POSH AND POSH-AP INHIBITORS

(75) Inventors: Philippe Nakache, Ness Ziona (IL); Nurit Livnah, Mazkeret Batya (IL)

(73) Assignee: PROTEOLOGICS, LTD., Rehovot (IL)

(21) Appl. No.: 12/674,675

(22) PCT Filed: Aug. 21, 2008
(86) PCT No.: PCT/IL2008/001146

§ 371 Date: Oct. 4, 2011

Related U.S. Application Data
(60) Provisional application No. 60/965,901, filed on Aug. 22, 2007.

(51) Int. Cl. A61K 31/505; A61K 31/44; A61P 31/12; A61P 31/14; A61P 25/24; A61P 31/20; A61P 25/00; A61P 25/28; A61P 25/16; A61P 25/18; C07D 213/76; A61P 31/18

(52) U.S. Cl. 514/275; 546/312; 514/352

(57) ABSTRACT

Pyridine and pyrimidine derivatives inhibit ubiquitination of human polypeptides, particularly of POSH and POSH-associated proteins such as HERPUD1, and can be used as medicaments for treatment of viral infections caused by virus such as HIV and for treatment of neurological disorders or diseases.

### FIELD OF THE INVENTION

The present invention relates to small molecule inhibitors of the ubiquitin ligase activity of a human polypeptide, particularly to POSH inhibitors, and to compositions and methods for treatment of viral infections and neurological conditions, disorders or diseases.

### BACKGROUND OF THE INVENTION

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However, the discovery of relevant gene or protein targets is often difficult and time consuming.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Viral maturation involves the proteolytic processing of the Gag proteins and the activity of various host proteins. It is believed that cellular machineries for exo/endocytosis and for ubiquitin conjugation may be involved in the maturation. In particular, the assembly, maturation, budding and subsequent release of retroid viruses, RNA viruses and envelope viruses, such as various retroviruses, rhabdoviruses, lentiviruses, and filoviruses may involve the Gag polyprotein. After its synthesis, Gag is targeted to the plasma membrane where it induces budding of nascent virus particles.

The role of ubiquitin in virus assembly was suggested by Dunigan et al. (1988, Virology 165, 310; Meyers et al. 1991, Virology 180, 602), who observed that mature virus particles were enriched in unconjugated ubiquitin. More recently, it was shown that proteasome inhibitors suppress the release of HIV-1, HIV-2 and virus-like particles derived from SIV and RSV Gag. Also, inhibitors affect Gag processing and maturation into infectious particles (Schubert et al 2000, PNAS 97, 13057; Harty et al. 2000, PNAS 97, 13871; Strack et al. 2000, PNAS 97, 13063; Patnaik et al. 2000, PNAS 97, 13069).

It is well known in the art that ubiquitin-mediated proteolysis is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell 2 surface receptors, biogenesis of ribosomes, and DNA repair. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and in some instances may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin may then be transferred to a specific cysteine on one of several E2 enzymes. Finally, these E2 enzymes donate ubiquitin to protein substrates, typically with the assistance of a E3 protein, also known as a ubiquitin ligase enzyme. In certain instances, substrates are recognized directly by the ubiquitin-conjugated E2 enzyme. Ubiquitin (ub) protein ligases (E3's) are functionally defined as proteins that facilitate the covalent linkage (conjugation) of one or multiple ubiquitin molecules to a substrate protein in the presence of E1 (ub-activating enzyme) and an E2 (ub carrier protein). In the absence of a protein substrate, E3's can catalyze self-ubiquitination, that is, transfer of activated ubiquitin from E2 to a lysine residue acceptor site on the E3 polypeptide, a reaction termed self-ubiquitination. Similar to trans ubiquitination, self-ubiquitination is dependent on the presence of E1, E2 and an intact E3 functional module i.e. RING finger or HECT domain (Lorick K L et al., Proc Natl Acad Sci USA. 1999 96:11364-9; Kao W H et al., J Virol. 2000 74:6408-6417).

It is also known that the ubiquitin system plays a role in a wide range of cellular processes including intracellular transport, cell cycle progression, apoptosis, and turnover of many membrane receptors. In viral infections, the ubiquitin system is involved not only with assembly, budding and release, but also with repression of host proteins such as p53, which may lead to a viral-induced neoplasm. The HIV Vpu protein interacts with an E3 protein that regulates IκB degradation, and is thought to promote apoptosis of infected cells by indirectly inhibiting NF-κB activity (Bour et al. (2001) J Exp Med 194:1299-311; U.S. Pat. No. 5,932,425). The ubiquitin system regulates protein function by both monoubiquitination and polyubiquitination. Polyubiquitination is primarily associated with protein degradation.

POSH (Plenty of SH3 domains) proteins play a role in a wide range of cellular processes including protein degradation, intracellular transport, cell cycle progression, apoptosis, and turnover of many membrane receptors. The essential function of POSH, a ubiquitin ligase, and “POSH proteins” (proteins that inherently include in their amino acid sequence a RING domain and at least one SH3 domain) in viral infection and the use of POSH inhibition to inhibit viral infections and, in particular, HIV infection, were broadly described in previous applications of the same applicant: WO 03/095971, WO 03/078601, WO 03/060067 and EP 1310552. All these applications are hereby incorporated by reference herein in their entirety as if fully disclosed herein.

An ubiquitin ligase, such as POSH, may participate in biological processes including, for example, one or more of the various stages of a viral lifecycle, such as viral entry into a cell, production of viral proteins, assembly of viral proteins and release of viral particles from the cell. In the patent applications mentioned hereinabove, it has been described that certain POSH polypeptides are involved in viral maturation, including the production, post-translational processing, assembly and/or release of proteins in a viral particle. Accordingly, viral infections may be ameliorated by inhibiting an activity (e.g. ubiquitin ligase activity or target protein interaction) of POSH.

In addition, as described in the applicant's WO 2004/078130, herein incorporated by reference in its entirety as if fully disclosed herein, several proteins interact with POSH and may be used to identify candidate therapeutics. One of these POSH-associated proteins (POSH-APs) is HERPUD1, known to be associated with neurological disorders, and in particular with Alzheimer's disease.

It would be beneficial to identify compounds as small molecules that bind POSH proteins and inhibit POSH protein activity and, more specifically, compounds that inhibit POSH protein-mediated ubiquitination.

Throughout this specification, various scientific publications and patents or published patent applications are referenced. The disclosure of all these publications in their entireties is hereby incorporated by reference into this specification in order to more fully describe the state of the art to which this invention pertains. Citation or identification of any reference in this section or any other part of this application shall not be construed as an admission that such reference is available as prior art to the invention.

### SUMMARY OF THE INVENTION

In one aspect, the present invention relates to the use of compounds of the general formula I as depicted hereinafter, for the preparation of a medicament. In preferred embodiments, the compounds of formula I are the compounds herein designated Compounds 1 and 2.

In a preferred embodiment, a compound of formula I is used according to the invention for inhibition of the ubiquitin ligase activity of a human polypeptide.

In a preferred embodiment, said human polypeptide contains a RING domain and, more preferably, further comprises at least one SH3 domain. In a most preferred embodiment, said polypeptide is a human POSH polypeptide.

The compounds of the general formula I were found to inhibit POSH protein-mediated ubiquitination, and are herein designated “POSH inhibitors”. They also inhibit ubiquitination of a POSH-associated protein (POSH-AP), such as HERPUD1, and thus are also POSH-AP inhibitors.

POSH polypeptides have been identified as playing a role in various stages of a virus lifecycle, including viral maturation, and also in neurological disorders. Thus, inhibition of a POSH polypeptide activity, in particular, POSH protein-mediated ubiquitination, will abolish such activities and will lead to treatment of a viral infection and, eventually, to viral death, or to treatment of a neurological condition, disorder or disease.

In one embodiment, the invention relates to a compound of formula I for use in the treatment of viral infections.

In another embodiment, the invention relates to a compound of formula I for use in the treatment of neurological conditions, disorders or diseases.

The invention further provides pharmaceutical compositions comprising a compound of formula I and a pharmaceutically acceptable carrier, particularly for treatment of viral infections or neurological conditions, disorders or diseases.

In another aspect, the present invention relates to a method for inhibiting the ubiquitin ligase activity of a human polypeptide, which comprises administering to a subject in need a compound of formula I in an amount effective for inhibiting the ubiquitin ligase activity of said human polypeptide.

In a further aspect, the present invention relates to a method for treatment of a patient suffering from a viral infection, particularly a viral infection caused by a retroid virus, an RNA virus and an envelop virus, including HIV, Ebola, HBV, HCV and HTLV, which comprises administering to said patient an effective amount of at least one compound of the general formula I hereinafter.

The present invention further provides the novel compound herein designated as Compound 2 of the formula depicted in Scheme 2 herein and pharmaceutical compositions comprising said compound.

### BRIEF DESCRIPTION OF THE FIGURES

FIG. 1 is a graph showing the antiviral effect of Compound 1 on CEM-SS cells infected with HIV-1IIIB virus, and the cytotoxic effect of Compound 1 on uninfected CEM-SS cells.

FIG. 2 is a graph showing the antiviral effect of Compound 2 on CEM-SS cells infected with HIV-1IIIB virus, and its cytotoxic effect on uninfected CEM-SS cells.

FIG. 3 is a graph showing the antiviral effect of Compound 2 on CEM-SS cells infected with HIV-2ROD virus, and the cytotoxic effect of Compound 2 on uninfected CEM-SS cells.

### DETAILED DESCRIPTION OF THE INVENTION

According to one aspect, the present invention provides a compound of the general formula I:

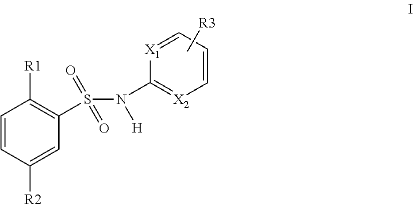

wherein

X₁ is C or N and X₂ is N;

R₁ and R₂ each independently is halogen, alkyl, aryl, heteroaryl, —COR₄, —COOR₄, —NR₅R₆, —CONR₅R₆ or —NR₇COR₈;

R₃ represents H or one to three radicals selected from lower alkyl, lower alkoxy, halogen, —COOR₄, —NR₅R₆ or —CONR₅R₆;

R₄ is H, hydrocarbyl or heterocyclyl;

R₅ and R₆ each independently is H, hydrocarbyl or heterocyclyl, or R₅ and R₆ together with the nitrogen atom to which they are attached form a saturated heterocyclic ring, optionally containing 1 or 2 further heteroatoms selected from N, S and/or O, and wherein said further N atom may be optionally substituted by lower alkyl optionally substituted by phenyl, halogen or hydroxy;

R₇ is H, lower alkyl or phenyl;

R₈ is aryl or heteroaryl;

wherein said lower alkyl, hydrocarbyl, heterocyclyl, aryl and heteroaryl is optionally substituted by one or more radicals selected from halogen, OR₄, —SR₄, —COR₄, —COOR₄, —NR₅R₆, —CONR₅R₆, —NO₂, —NR₅—COR₄ —SO₃R₄, —SO₂R₄, —SO₂NR₅R₆ and —NR₅SO₂R₄, wherein R₄, R₅ and R₆ are as defined above;

or an enantiomer or a pharmaceutically acceptable salt thereof, for use as a medicament.

Without limiting the scope to further possible definitions, as used herein in the specification, the terms hereinbelow are defined as follows:

The term “hydrocarbyl” means a radical derived from a straight or branched acyclic or cyclic, saturated or unsaturated, including aromatic, hydrocarbon, and includes alkyl, alkenyl, alkynyl, cycloalkyl, cycloalkenyl, aralkyl and aryl. The terms “alkyl”, “alkenyl”, or “alkynyl” refer to a C₁-C₁₀, preferably C₁-C₄, alkyl, C₂-C₁₀, preferably C₂-C₄, alkenyl, or C₂-C₁₀, preferably C₂-C₄, alkynyl, respectively, that may be straight or branched and may be interrupted by one or more heteroatoms selected from O, S and/or N, and/or substituted by one or more radicals selected from the group consisting of halogen, aryl, heterocyclyl, nitro, epoxy, epithio, OR′, —SR′, —COR′, —COOR′ —NR′R″, —CONR′R″, —NR′COR″ —SO₃R′, —SO₂R′, —SO₂NR′R″ and —NR′SO₂R′, wherein R′ and R″, independently, each is H, hydrocarbyl or heterocyclyl, or R′ and R″ together with the nitrogen atom to which they are attached form a saturated heterocyclic ring, optionally containing 1 or 2 further heteroatoms selected from N, S and/or O, and wherein said further N atom may be optionally substituted by hydrocarbyl.

Examples of lower alkyl include, without being limited to, methyl, ethyl, n-propyl, isopropyl, n-butyl, isobutyl, tert-butyl, and is preferably methyl. Any C₂-C₄ alkenyl may be straight or branched and may contain one or two double bonds, e.g. alkadienyl radical, and preferably a terminal double bond, and includes for example vinyl, prop-2-en-1-yl, but-3-en-1-yl. Any C₂-C₄ alkynyl may be straight or branched and may contain one or more triple bonds and includes, for example, ethynyl, propynyl, butynyl. All alkyl, alkenyl, and alkynyl radicals may be substituted as defined hereinabove.

The terms “cycloalkyl” and “cycloalkenyl” refer to a C₅-C₆ cycloalkyl or C₅-C₆ cycloalkenyl, respectively, and include, without being limited to, cyclopentyl, cyclohexyl, cyclopentenyl and cyclohexenyl, that may be substituted by one or more radicals as defined hereinabove.

The term “aryl” refers to a C₆-C₁₄ aromatic carbocyclic group having 6 to 14 carbon atoms, preferably 6 to 10 carbon atoms, consisting of a single, bicyclic or tricyclyc rings, such as phenyl, naphthyl and antracenyl that may be substituted by one or more radicals as defined hereinabove. The term “aralkyl” refers to a radical derived from an arylalkyl compound and includes benzyl, phenethyl and the like.

The term “heterocyclyl” means a radical derived from a saturated, partially unsaturated, optionally substituted, monocyclic, bicyclic or tricyclic heterocycle of 3-12, preferably 5-10, more preferably 5-6 members in the ring containing 1 to 3 heteroatoms selected from O, S and/or N. Particular examples are, without being limited to, dihydrofuryl, tetrahydrofuryl, pyrrolynyl, pyrrolydinyl, dihydrothienyl, dihydropyridyl, piperidinyl, quinolinyl, piperazinyl, morpholino or 1,3-dioxanyl.

The term “heteroaryl” refers to a mono- or polycyclic heteroaromatic ring containing 1 to 3 heteroatoms selected from O, S and/or N and optionally substituted. Particular examples are, without being limited to, pyrrolyl, furyl, thienyl, pyrazolyl, imidazolyl, oxazolyl, thiazolyl, pyridyl, quinolinyl, isoquinolinyl, pyridazinyl, pyrimidinyl, pyrazinyl, 1,3,4-triazinyl, 1,2,3-triazinyl, 1,3,5-triazinyl, benzofuryl, isobenzofuryl, indolyl, imidazo[1,2-a]pyridyl, benzimidazolyl, benzthiazolyl, benzoxazolyl, benzodiazepinyl, and other radicals derived from further polycyclic heteroaromatic rings. The heterocyclyl radical may be substituted by one or more radicals as defined herein above. It is to be understood that when a polycyclic heterocyclic ring is substituted, the substitutions may be in any of the carbocyclic and/or heterocyclic rings.

The term “halogen” refers to fluoro, chloro, bromo or iodo. In preferred embodiments, the halogen is chloro.

The groups —NR′R″ or —NR₄R₅ may be —NH₂, when R′ and R″ or R₄ and R₅, respectively, are both hydrogen, or may be a secondary amino group when R′ (or R₄) is H and R″ (or R₅) is C₁-C₄ alkyl, or tertiary amino when R′ (or R₄) and R″ (or R₅) are each C₁-C₄ alkyl, or R′ and R″ (R₄ or R₅, respectively) together with the nitrogen atom to which they are attached may form a saturated, 5-7 membered, preferably 5- or 6-membered, heterocyclic ring, optionally containing 1 or 2 further heteroatoms selected from nitrogen, oxygen and/or sulfur. Such rings may be substituted, for example with one or two alkyl groups, preferably at a further N atom. Examples of such rings include, without being limited to, pyrrolidino, piperidino, morpholino, thiomorpholino, piperazino, N-alkylpiperazino, e.g. N-methylpiperazino, and diazepino.

Any alkoxy, alkylthio or alkanoyl groups formed by the radicals OR₄, OR′, SR₄, SR′, COR₄ and COR′, when R₄ or R′ is alkyl, are preferably C₁-C₄ alkoxy, C₁-C₄ alkylthio and C₂-C₄ alkanoyl groups, respectively. Examples of alkoxy are methoxy, ethoxy, propyloxy, butoxy, and the like, examples of alkylthio are methylthio, ethylthio, propylthio, butylthio, and examples of alkanoyl are acetyl, propanoyl, butanoyl, and the like. All alkoxy, thioalkyl, and alkanoyl radicals may be substituted as defined above. In one preferred embodiment, the C₁-C₄ alkoxy is methoxy.

Also contemplated by the present invention is the use of salts of the compounds of formula I, including salts formed by any carboxy or sulfo groups present in the molecule and a base as well as acid addition and/or base salts.

Pharmaceutically acceptable salts are formed with metals or amines, such as alkali and alkaline earth metals or organic amines. Examples of metals are sodium, potassium, magnesium, calcium, and the like, and examples of suitable amines are N,N′-dibenzylethylenediamine, chloroprocaine, choline, diethanolamine, ethylene-diamine, N-methylglucamine, and procaine (see, for example, Berge S. M., et al., “Pharmaceutical Salts,” (1977) J. of Pharmaceutical Science, 66:1-19). The salts can also be pharmaceutically acceptable quaternary salts such as a quaternary salt of the formula —NRR′R″+Z′ wherein R, R′and R″ each is independently hydrogen, alkyl or benzyl and Z is a counterion, including chloride, bromide, iodide, O-alkyl, toluenesulfonate, methylsulfonate, sulfonate, phosphate, carboxylate, acetate or trifluoroacetate.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, acetate, trifluoroacetate and the like. Also contemplated are salts of amino acids such as arginate and the like and gluconate or galacturonate (see, for example, Berge S. M., et al., “Pharmaceutical Salts,” (1977) J. of Pharmaceutical Science, 66:1-19).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

In one preferred embodiment of the present invention, the compounds of formula I are pyridine compounds, i.e., X₁ is C and X₂ is N, or pyrimidine compounds, i.e., X₁ and X₂ are both N, preferably pyridine and pyrimidine compounds of the general formula I wherein R₁ is halogen, R₂ is halogen, alkyl, aryl, heteroaryl, —COR₄, —COOR₄, —NR₅R₆, —CONR₅R₆ or —NR₇COR₈, and R₃ represents two lower alkyl radicals, more preferably, R₁ and R₂ are both halogen, most preferably, Cl, and R₃ represents two methyl radicals

In a more preferred embodiment, the compound is the pyrimidine compound herein identified as Compound 1, wherein R₁ and R₂ each is —Cl, R₃ represents two methyl groups at positions 4 and 6, and X₁ and X₂ each is a nitrogen atom. In another more preferred embodiment, the compound is the novel pyridine compound herein identified as Compound 2, wherein R₁ and R₂ each is —Cl, R₃ represents two methyl groups at positions 4 and 6, X₁ is C and X₂ is a nitrogen atom. These compounds are identified in the description, Examples and in the Claims by their number in bold. The structural formulas of Compounds 1 and 2 are presented in Appendix A hereinafter in the specification (after the Scheme, just before the claims).

Compound 2 was prepared, in accordance with the present invention, using a one-step reaction procedure as described in Example 8 and Scheme 1.

It has been discovered, according to the present invention, that the compounds of formula I herein are POSH protein inhibitors.

The terms “POSH” or “POSH protein(s)” or “POSH polypeptide(s)” are used herein interchangeably and refer to a polypeptide that includes in its amino acid sequence a RING domain and at list one SH3 domain. In some instances, the POSH protein may have 3 or 4 SH3 domains.

The terms “POSH-mediated ubiquitination” or “POSH protein-mediated ubiquitination” are used interchangeably and refer to any ubiquitination process that requires the involvement of a POSH protein.

The terms “POSH inhibitor” or “POSH protein inhibitor” are used interchangeably and refer to a chemical compound that inhibits a POSH activity as defined in WO 03/095971, hereby incorporated by reference in its entirety as if fully disclosed herein, including POSH protein-mediated ubiquitination.

Thus, in one preferred embodiment, the invention relates to a POSH protein inhibitor for use as a medicament, wherein said POSH protein inhibitor is a compound of a general formula I above. All the preferred embodiments defined above relating to use of a compound of formula I apply also to the use of a POSH inhibitor.

In a preferred embodiment, the POSH protein inhibitors of the general formulas I inhibit the ubiquitin ligase activity of a POSH protein. In another preferred embodiments, the POSH inhibitors inhibit POSH selfubiquitination, particularly the RING-finger dependent ubiquitination of the POSH protein. In a more preferred embodiment, the POSH inhibitors inhibit POSH selectively and do not inhibit the FINGER-dependent ubiquitination of other ubiquitin E3 ligases such as Mdm2 and c-Cb1 that have no SH3 domain.

In a most preferred embodiment, the invention provides the use of compounds of the general formula I for the preparation of a medicament exhibiting antiviral activity.

In another aspect, the present invention relates to a method for treatment of a patient suffering from a viral infection, which comprises administering to said patient an effective amount of a compound of the general formula I hereinabove.

According to the invention, it is envisaged that the POSH protein inhibitors will be useful for the treatment of any viral infection.

In view of the teachings herein, one of skill in the art will understand that the methods and compositions of the invention are applicable to a wide range of viruses such as for example retroid viruses, RNA viruses, and envelope viruses. The term “envelope virus” as used herein refers to any virus that uses cellular membrane and/or any organelle membrane in the viral release process.

In some preferred embodiments, the present invention is applicable to retroid viruses and to lentivirus, including primate lentivirus. In a more preferred embodiment, the invention is applicable to retroviruses (retroviridae). While not intended to be limiting, relevant viruses include: C-type retrovirus which causes lymphosarcoma in Northern Pike, the C-type retrovirus which infects mink, the C-type retrovirus which infects pigs, the caprine lentivirus which infects sheep, equine infectious anemia virus (EIAV), avian leukosis sarcoma virus (ALSV), feline leukemia virus (FeLV), feline Aids virus, bovine leukemia virus (BLV), simian leukemia virus (SLV), simian immunodeficiency virus (SIV), human T-cell leukemia virus type-I (HTLV-I), human T-cell leukemia virus type-II (HTLV-II), human immunodeficiency virus type-2 (HIV-2) and human immunodeficiency virus type-1 (HIV-1).

The method and compositions of the present invention are further applicable to RNA viruses, including ssRNA negative-strand viruses and ssRNA positive-strand viruses. The ssRNA positive-strand viruses include hepatitis C virus (HCV). In a preferred embodiment, the present invention is applicable to mononegavirales, including filoviruses. Filoviruses further include Ebola viruses and Marburg viruses.

Other RNA viruses include picornaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus. Additional viruses that may be treated include herpes viruses.

In preferred features according to preferred embodiments of the invention, the viral infection is caused by a virus selected from human immunodeficiency virus (HIV), particularly HIV-1 or HIV-2, Ebola virus, hepatitis B virus (HBV), hepatitis C virus (HCV) and a human T-cell leukemia virus (HTLV) such as HTLV type 1 (HTLV-1).

In a most preferred embodiment of the present invention, the compound used for treatment of viral infections, preferably of viral infections caused by a HIV virus, is the compound herein identified as Compound 1. In another most preferred embodiment, the compound used for treatment of HIV infection is herein identified as Compound 2.

In another most preferred embodiment, the invention provides pharmaceutical compositions for treatment of neurological conditions, disorders or diseases comprising a pharmaceutically acceptable carrier and a compound of a general formula I.

In still another aspect, the present invention relates to a method for treatment of a patient suffering from a neurological condition, disorder or disease, which comprises administering to said patient an effective amount of at least one compound of a general formula I hereinabove.

According to the present invention, any neurological condition, disorder or disease may be treated with the compounds of formula I including, without limitation, Alzheimer's disease, Parkinson's disease, Huntington's disease, Pick's disease, cerebral vascular disease, depression or schizophrenia.

In a preferred feature according to a preferred embodiment of the invention, the neurological disease is Alzheimer's disease. According to this feature, the present invention provides a method of inhibiting amyloid polypeptide production in a cell comprising administering a small molecule agent that inhibits the ubiquitin ligase activity of a human polypeptide or protein, wherein said small molecule compound is a compound of the general formula I herein. In another embodiment, the invention provides a method of inhibiting the transport of amyloid precursor protein (APP) in a cell comprising inhibiting the ubiquitin ligase activity of a polypeptide with a small molecule, wherein said small molecule compound is a compound of formula I in claim 1.

The pharmaceutical compositions for use in accordance with the present invention may be formulated in conventional manner using one or more physiologically acceptable carriers or excipients. Thus, the compounds and their physiologically acceptable salts and solvates may be formulated by conventional methods as described, for example, in Remington's Pharmaceutical Sciences, Meade Publishing Co., Easton, Pa., for administration by a variety of routes of administration, including systemic and topical or localized administration.

For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents; fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets, pills, capsules or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetra-fluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compounds of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

POSH interacts with and regulates a wide range of key cellular functions that may be manipulated by affecting the level of and/or activity of POSH polypeptides or POSH-AP polypeptides. Many features of POSH, and particularly human POSH, are described in WO 03/095971 and WO 03/078601.

As described in the above-referenced publications, native human POSH is a large polypeptide containing a RING domain and four SH3 domains. POSH is a ubiquitin ligase (also termed an “E3” enzyme); the RING domain mediates ubiquitination of, for example, the POSH polypeptide itself. POSH interacts with a large number of proteins and participates in a host of different biological processes. As demonstrated in this disclosure, POSH associates with a number of different proteins in the cell. POSH co-localizes with proteins that are known to be located in the trans-Golgi network, implying that POSH participates in the trafficking of proteins in the secretory system. The term “secretory system” should be understood as referring to the membrane compartments and associated proteins and other molecules that are involved in the movement of proteins from the site of translation to a location within a vacuole, a compartment in the secretory pathway itself, a lysosome or endosome or to a location at the plasma membrane or outside the cell. Commonly cited examples of compartments in the secretory system include the endoplasmic reticulum, the Golgi apparatus and the cis and trans Golgi networks.

In addition, Applicants have demonstrated that POSH is necessary for proper secretion, localization or processing of a variety of proteins, including phospholipase D, HIV Gag, HIV Nef, Rapsyn and Src. Many of these proteins are myristoylated, indicating that POSH plays a general role in the processing and proper localization of myristoylated proteins. Accordingly, in certain aspects, POSH may play a role in the processing and proper localization of myristolyated proteins. N-myristoylation is an acylation process, which results in covalent attachment of myristate, a 14-carbon saturated fatty acid to the N-terminal glycine of proteins (Farazi et al., J. Biol. Chem. 276: 39501-04 (2001)). N-myristoylation occurs co-translationally and promotes weak and reversible protein-membrane interaction. Myristoylated proteins are found both in the cytoplasm and associated with membrane. Membrane association is dependent on protein configuration, i.e., surface accessibility of the myristoyl group may be regulated by protein modifications, such as phosphorylation, ubiquitination etc. Modulation of intracellular transport of myristoylated proteins in the application includes effects on transport and localization of these modified proteins.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a POSH polypeptide.

In certain embodiments, the agents of the invention identified are antiviral agents, optionally interfering with viral maturation, and preferably where the virus is a retroid virus, an RNA virus and an envelope virus.

In certain preferred embodiments, an antiviral agent interferes with the ubiquitin ligase (catalytic) activity of POSH (e.g. POSH auto-ubiquitination or transfer to a target protein).

In additional certain preferred embodiments, an antiviral agent interferes with the interaction between POSH and a POSH-AP (adaptor) polypeptide, for example an antiviral agent may disrupt or render irreversible the interaction between a POSH polypeptide and POSH-AP polypeptide such as another POSH polypeptide (as in the case of a POSH dimer, a heterodimer of two different POSH polypeptides, homomultimers and heteromultimers); a GTPase (eg. Rac, Rac1, Rho, Ras); an E2 enzyme and ubiquitin, or optionally, a cullin; a clathrin; AP-1; AP-2; an HSP70; an HSP90, Brca1, Bard1, Nef, PAK1, PAK2, PAK family, Vav, Cdc42, PI3K (e.g. p85 or p110), Nedd4, src (src family), a Gag, particularly an HIV Gag (e.g. p160), Tsg101, VASP, RNB6, WASP, N-WASP and KIAA0674, similar to Spred-2, as well as, in certain embodiments, proteins known to be associated with clathrin-coated vesicles and or proteins involved in the protein sorting pathway.

In yet additional embodiments, agents of the invention interfere with the signaling of a GTPase, such as Rac or Ras, optionally disrupting the interaction between a POSH polypeptide and a Rac protein.

In certain embodiments, agents of the invention modulate the ubiquitin ligase activity of POSH and may be used to treat certain diseases related to ubiquitin ligase activity.

In other certain embodiments, the invention discloses assays to identify, optimize or otherwise assess agents that decrease a ubiquitin-related activity of a POSH polypeptide. Ubiquitin-related activities of POSH polypeptides may include the self-ubiquitination activity of a POSH polypeptide, generally involving the transfer of ubiquitin from an E2 enzyme to the POSH polypeptide, and the ubiquitination of a target protein (e.g., HERPUD1), generally involving the transfer of a ubiquitin from a POSH polypeptide to the target protein. In certain embodiments, a POSH activity is mediated, at least in part, by a POSH RING domain.

In still other certain embodiments, an assay comprises forming a mixture comprising a POSH polypeptide, an E2 polypeptide and a source of ubiquitin (which may be the E2 polypeptide pre-complexed with ubiquitin). Optionally the mixture comprises an E1 polypeptide and optionally the mixture comprises a target polypeptide, such as, for example, HERPUD1. Additional components of the mixture may be selected to provide conditions consistent with the ubiquitination of the POSH polypeptide. One or more of a variety of parameters may be detected, such as POSH-ubiquitin conjugates, E2-ubiquitin thioesters, free ubiquitin and target polypeptide-ubiquitin complexes.

The term “detect” is used herein to include a determination of the presence or absence of the subject of detection (e.g. POSH-ubiquitin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g. fluorescent label, radioisotope label, and other described below), resolution and identification by size (e.g. SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the POSH-ubiquitin conjugate.

In certain embodiments, an assay comprises forming a mixture comprising a POSH polypeptide, a target polypeptide and a source of ubiquitin (which may be the POSH polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as POSH-ubiquitin conjugates and target polypeptide-ubiquitin conjugates. In a preferred embodiment, an assay comprises detecting the target polypeptide-ubiquitin conjugate, such as, for example, detecting ubiquitinated HERPUD1. In another preferred embodiment, an assay comprises detecting the POSH-ubiquitin conjugate.

An assay described above may be used in a screening assay to identify agents that modulate a ubiquitin-related activity of a POSH polypeptide. A screening assay will generally involve adding a test agent to one of the above assays, or any other assay designed to assess a ubiquitin-related activity of a POSH polypeptide. The parameter(s) detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred. For example, in certain embodiments, it may be desirable to pre-incubate the test agent and the E3 (e.g. the POSH polypeptide), followed by removing the test agent and addition of other components to complete the assay. In this manner, the effects of the agent solely on the POSH polypeptide may be assessed. In certain preferred embodiments, a screening assay for an antiviral agent employs a target polypeptide comprising an L domain, and preferably an HIV L domain.

In certain embodiments, an assay is performed in a high-throughput format. For example, one of the components of a mixture may be affixed to a solid substrate and one or more of the other components is labeled. For example, the POSH polypeptide may be affixed to a surface, such as a 96-well plate, and the ubiquitin is in solution and labeled. An E2 and E1 are also in solution, and the POSH-ubiquitin conjugate formation may be measured by washing the solid surface to remove uncomplexed labeled ubiquitin and detecting the ubiquitin that remains bound. Other variations may be used. For example, the amount of ubiquitin in solution may be detected.

In certain embodiments, the formation of ubiquitin complexes may be measured by an interactive technique, such as FRET, wherein a ubiquitin is labeled with a first label and the desired complex partner (e.g. POSH polypeptide or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiquitin (see, e.g. US Patent Publication 20020042083). High-throughput screening may be achieved by performing an interactive assay, such as FRET, in solution as well. In addition, if a polypeptide in the mixture, such as the POSH polypeptide or target polypeptide, is readily purifiable (e.g. with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As described above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g. epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids (see chapter 4, Molecular Probes Catalog, Haugland, 6th Ed. 1996, hereby incorporated by reference). A biotinylated substrate can be attached to a biotinylated component via avidin or streptavidin. Similarly, a large number of haptenylation reagents are also known.

In an alternative embodiment, a POSH polypeptide, E2 or target polypeptide is bound to a bead, optionally with the assistance of a tag. Following ligation, the beads may be separated from the unbound ubiquitin and the bound ubiquitin measured. In a preferred embodiment, POSH polypeptide is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated by reference in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is used at a final concentration of from 5 to 200 ng per 100 microliter reaction solution. Optionally E1 is used at a final concentration of from 1 to 50 ng per 100 microliter reaction solution. Optionally E2 is used at a final concentration of 10 to 100 ng per 100 microliter reaction solution, more preferably 10-50 ng per 100 microliter reaction solution. In a preferred embodiment, POSH polypeptide is used at a final concentration of from 1 ng to 500 ng per 100 microliter reaction solution.

Generally, an assay mixture is prepared so as to favor ubiquitin ligase activity and/or ubiquitination activity. Generally, this will be physiological conditions, such as 50-200 mM salt (e.g. NaCl, KCl), pH of between 5 and 9, and preferably between 6 and 8. Such conditions may be optimized through trial and error. Incubations may be performed at any temperature which facilitates optimal activity, typically between 4 and 40 degrees C. Incubation periods are selected for optimum activity, but may also be optimized to facilitate high through put screening. Typically between 0.5 and 1.5 hours will be sufficient.

A variety of other reagents may be included in the compositions. These include reagents like salts, solvents, buffers, neutral proteins, e.g. albumin, detergents, etc. which may be used to facilitate optimal ubiquitination enzyme activity and/or reduce non-specific or background interactions.

Also reagents that otherwise improve the efficiency of the assay, such as protease inhibitors, nuclease inhibitors, anti-microbial agents, etc., may be used. The compositions will also preferably include adenosine tri-phosphate (ATP).

The mixture of Components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a POSH ubiquitin-related activity may be used to inhibit POSH function in vivo. The test agent may be modified for use in vivo, e.g. by addition of a hydrophobic moiety, such as an ester.

Certain embodiments of the invention relate to assays for identifying agents that bind to a POSH polypeptide or POSH-AP, such as HERPUD1, or optionally, a particular domain of POSH such as an SH3 or RING domain. In preferred embodiments, a POSH polypeptide is a polypeptide comprising the fourth SH3 domain of hPOSH (SEQ ID NO: 30 as described in). A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject POSH polypeptides with a POSH-AP. In another embodiment, the assay detects agents, which modulate the intrinsic biological activity of a POSH polypeptide or POSH complex, such as an enzymatic activity, binding to other cellular components, cellular compartmentalization, and the like.

In one aspect, the invention provides methods and compositions for the identification of compositions that interfere with the function of POSH polypeptides or POSH-AP, such as HERPUD1. Given the role of POSH polypeptides in viral production, compositions that perturb the formation or stability of the protein-protein interactions between POSH polypeptides and the proteins that they interact with, such as POSH-APs, and particularly POSH complexes comprising a viral protein, are candidate pharmaceuticals for the treatment of viral infections.

While not wishing to be bound to mechanism, it is postulated that POSH polypeptides promote the assembly of protein complexes that are important in release of virions and other biological processes. Complexes of the invention may include a combination of a POSH polypeptide and one or more of the following POSH-APs: a POSH-AP; a POSH polypeptide (as in the case of a POSH dimer, a heterodimer of two different POSH, homomultimers and heteromultimers); Vpu; Cb1-b; PKA; UNC84; MSTP028; HERPUD1; GOCAP1; PTPN12; EIF3S3; SAR1; GOSR2; RALA; SIAH; SMIN1; SMN2; SYNE1; TTC3; VCY2IP1; SAM68; gag-pol; a GTPase an E2 enzyme; ubiquitin, or optionally, a cullin; a clathrin; AP-1; AP-2; an HSP70; an HSP90, Brca1, Bard1, Nef, PAK1, PAK2, PAK family, Vav, Cdc42, PI3K (e.g. p85 or p110), Nedd4, src (src family), Tsg101, VASP, RNB6, WASP, N-WASP, a Gag, particularly an HIV Gag (e.g. p160); and KIAA0674, Similar to Spred-2, as well as, in certain embodiments, proteins known to be associated with clathrin-coated vesicles and or proteins involved in the protein sorting pathway.

The type of complex formed by a POSH polypeptide will depend upon the domains present in the protein. While not intended to be limiting, exemplary domains of potential interacting proteins are provided below. A RING domain is expected to interact with cullins, E2 enzymes, AP-1, AP-2, and/or a substrate for ubiquitylation (e.g. in some instances, a protein comprising a Gag L domain or a Gag polypeptide such as Gag-Pol, such as HIV p160). An SH3 domain may interact with Gag L domains and other proteins having the sequence motifs as disclosed in WO03/095971, the teachings of which are incorporated by reference herein.

In a preferred assay for an antiviral agent, the test agent is assessed for its ability to disrupt or inhibit the formation of a complex of a POSH polypeptide and a Rac polypeptide, particularly a human Rac polypeptide, such as Rac 1.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a POSH polypeptide-mediated membrane reorganization or vesicle formation activity, may be generated in many different forms, and include assays based on cell-free systems, e.g. purified proteins or cell lysates, as well as cell-based assays which utilize intact cells. Simple binding assays can also be used to detect agents, which bind to POSH. Such binding assays may also identify agents that act by disrupting the interaction between a POSH polypeptide and a POSH interacting protein, or the binding of a POSH polypeptide or complex to a substrate. Agents to be tested can be produced, for example, by bacteria, yeast or other organisms (e.g. natural products), produced chemically (e.g. small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug-screening programs, which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifested in an alteration of binding affinity with other proteins or changes in enzymatic properties of the molecular target.

In preferred embodiments of the present assay, a reconstituted POSH complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in POSH complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin), which might interfere with or otherwise alter the ability to measure POSH complex assembly and/or disassembly.

Assaying POSH complexes, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with assembly or stability of the POSH complex. In an exemplary binding assay, the compound of interest is contacted with a mixture comprising a POSH polypeptide and at least one interacting polypeptide. Detection and quantification of POSH complexes provides a means for determining the compound's efficacy at inhibiting interaction between the two polypeptides. The efficacy of the compound can be assessed by generating dose response Curves from data obtained using various concentrations of the test compound. Moreover, a control assay can also be performed to provide a baseline for comparison. In the control assay, the formation of complexes is quantitated in the absence of the test compound.

Complex formation between the POSH polypeptides and a substrate polypeptide may be detected by a variety of techniques, many of which are effectively described above. For instance, modulation of the formation of complexes can be quantitated using, detectably labeled proteins (e.g. radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-POSH fusion proteins can be adsorbed onto glutathione sepharose beads (Sigma Chemical, St. Louis, Mo.) or glutathione derivatized microtitre plates, which are then combined with a potential interacting protein, e.g. an 35S-labeled polypeptide, and the test compound and incubated under conditions conducive to complex formation. Following incubation, the beads are washed to remove any unbound interacting protein, and the matrix bead-bound radiolabel determined directly (e.g. beads placed in scintillant), or in the supernatant after the complexes are dissociated, e.g. when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In another embodiment, the POSH polypeptide and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In still further embodiments of the present assay, the POSH complex is generated in whole cells, taking advantage of cell culture techniques to support the subject assay. For example, as described below, the POSH complex can be constituted in a eukaryotic cell culture system, including mammalian and yeast cells. Often it will be desirable to express one or more viral proteins (eg. Gag or Env) in such a cell along with a subject POSH polypeptide. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high-throughput analysis of candidate agents.

The components of the POSH complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a POSH activity. In certain embodiments a POSH activity is represented by production of virus like particles. As demonstrated herein, an agent that disrupts POSH activity can cause a decrease in the production of virus like particles. In certain embodiments, POSH activities may include, without limitation, complex formation, ubiquitination and membrane fusion events (eg. release of viral buds or fusion of vesicles). POSH complex formation may be assessed by immunoprecipitation and analysis of co-immunoprecipitated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein (CFP) is excited by light at roughly 425-450 nm wavelength and emits light in the range of 450-500 nm. Yellow fluorescent protein (YFP) is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide may be measured.

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570; Squire et al. (1999) J. Microsc. 193: 36; Verveer et al. (2000) Biophys. J. 78 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, the POSH polypeptide and the interacting protein of interest are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow or red (termed BFP, CFP, YFP and RFP, respectively). Fluorescent proteins may be stably attached to polypeptides through a variety of covalent and noncovalent linkages, including, for example, peptide bonds (eg. expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol., 35:803-808; Levine et al. (1982) Comp. Biochem. Physiol., 72B; 77-85; Tersikh et al. (2000) Science 290: 1585-88.

Other exemplary fluorescent moieties well known in the art include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.), along with methodologies for modifying polypeptides with such moieties. Exemplary proteins that fluoresce when combined with a fluorescent moiety include, yellow fluorescent protein from Vibrio fischeri (Baldwin et al. (1990) Biochemistry 29:5509-15), peridinin-chlorophyll a binding protein from the dinoflagellate Symbiodinium sp. (Morris et al. (1994) Plant Molecular Biology 24:673:77) and phycobiliproteins from marine cyanobacteria such as Synechococcus, e.g., phycoerythrin and phycocyanin (Wilbanks et al. (1993) J. Biol. Chem. 268:1226-35). These proteins require flavins, peridinin-chlorophyll a and various phycobilins, respectively, as fluorescent co-factors.

FRET-based assays may be used in cell-based assays and in cell-free assays. FRET-based assays are amenable to high-throughput screening methods including Fluorescence Activated Cell Sorting and fluorescent scanning of microtiter arrays.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

In further embodiments, the invention provides methods for identifying targets for therapeutic intervention. A polypeptide that interacts with POSH or participates in a POSH-mediated process (such as viral maturation) may be used to identify candidate therapeutics. Such targets may be identified by identifying proteins that associate with POSH (POSH-APs) by, for example, immuno-precipitation with an anti-POSH antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay.

In particular, the yeast two-hybrid screen makes use of chimeric genes, which express hybrid proteins. To illustrate, a first hybrid gene comprises the coding sequence for a DNA-binding domain of a transcriptional activator can be fused in frame to the coding sequence for a “bait” protein, e.g., a POSH polypeptide of sufficient length to bind to a potential interacting protein. The second hybrid protein encodes a transcriptional activation domain fused in frame to a gene encoding a “fish” protein, e.g., a potential interacting protein of sufficient length to interact with the POSH polypeptide portion of the bait fusion protein. If the bait and fish proteins are able to interact, e.g., form a POSH complex, they bring into close proximity the two domains of the transcriptional activator. This proximity causes transcription of a reporter gene, which is operably linked to a transcriptional regulatory site responsive to the transcriptional activator, and expression of the reporter gene can be detected and used to score for the interaction of the bait and fish proteins.

Targets that have been identified by such approaches include HERPUD1. Other targets that may be identified by such approaches include: Vpu; Cb1-b; PKA; UNC84; MSTP028; GOCAP1; PTPN12; EIF3S3; SAR1; GOSR2; RALA; SIAH; SMIN1; SMN2; SYNE1; TTC3; VCY2IP1; SAM68; Gag-Pol; a GTPase a GTPase (eg. Rac, Rac1, Rho, Ras); an E2 enzyme, a cullin; a clathrin; AP-1; AP-2; an HSP70; an HSP90, Brca1, Bard1, Nef, PAK1, PAK2, PAK family, Vav, Cdc42, PI3K (e.g. p85 or p110), Nedd4, src (src family), Tsg101, VASP, RNB6, WASP, N-WASP, a Gag, particularly an HIV Gag (e.g. p160); and KIAA0674, Similar to Spred-2, as well as, in certain embodiments, proteins known to be associated with clathrin-coated vesicles, proteins involved in the protein sorting pathway and proteins involved in a Rac signaling pathway.

A variety of other reagents may be included in the screening assay. These include reagents like salts, neutral proteins, e.g. albumin, detergents, etc that are used to facilitate optimal protein-protein binding and/or reduce nonspecific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° and 40° C. Incubation periods are selected for optimum activity, but may also be optimized to facilitate rapid high-throughput screening.

In certain embodiments, a test agent may be assessed for its ability to perturb the localization of a POSH polypeptide, e.g. preventing POSH localization to the nucleus and/or the Golgi network.

In applicant's previous application PCT/US2004/10582 filed on Apr. 5, 2004, herein incorporated by reference in its entirety, the discovery of novel associations between POSH proteins and HERPUD1 proteins, and related methods and compositions were described. In said application, novel associations among certain disease states, POSH nucleic acids and proteins, and HERPUD1 nucleic acids and proteins, were also disclosed.

By identifying proteins associated with POSH, and particularly human POSH, the present application provides a conceptual link between the POSH-APs and cellular processes and disorders associated with POSH-APs, and POSH itself Accordingly, in certain embodiments of the disclosure, agents that modulate a POSH-AP, such as HERPUD1, may now be used to modulate POSH functions and disorders associated with POSH function, such as neurological disorders. Likewise, in certain embodiments of the disclosure, agents that modulate POSH may now be used to modulate POSH-AP, such as HERPUD1, functions and disorders associated with POSH-AP function, such as disorders associated with HERPUD1 function, including HERPUD1-associated neurological disorders. Additionally, test agents may be screened for an effect on HERPUD1 and then further tested for effect on a POSH-AP function or a disorder associated with POSH-AP function. In the PCT application mentioned above, it was disclosed that a POSH polypeptide interacts with one or more HERPUD1 polypeptides.

The term “amyloid polypeptide” is used to refer to any of the various polypeptides that are significant components of amyloid plaque as well as precursors thereof. The amyloid beta A4 precursor protein (“APP”) gives rise to smaller proteins, such as the roughly 40 amino acid beta-amyloid proteins that form a major component of the amyloid plaque associated with Alzheimer's disease, Down's syndrome (in older patients) and certain hereditary cerebral hemorrhage amyloidoses. APP has several isoforms generated by alternative splicing of a 19-exon gene: exons 1-13, 13a, and 14-18 (Yoshikai et al., 1990). The predominant transcripts are APP695 (exons 1-6, 9-18, not 13a), APP751 (exons 1-7, 9-18, not 13a), and APP770 (exons 1-18, not 13a). All of these encode multidomain proteins with a single membrane-spanning region. They differ in that APP751 and APP770 contain exon 7, which encodes a serine protease inhibitor domain. APP695 is a predominant form in neuronal tissue, whereas APP751 is the predominant variant elsewhere. Beta-amyloid is derived from that part of the protein encoded by parts of exons 16 and 17. All of the isoforms of APP and any of the smaller proteins derived therefrom are included in the term “amyloid polypeptide”, as well as any of the various naturally occurring variations thereof and any artificially produced variants that retain one or more functional properties of the naturally occurring protein or that are useful as a proxy for monitoring the production of APP or a protein derived therefrom. The subset of amyloid polypeptides that are APP or derived therefrom may be referred to specifically as “APP amyloid polypeptides”. Yoshikai et al. Gene 87: 257-263, 1990.

A “POSH-associated protein” or “POSH-AP” refers to a protein capable of interacting with and/or binding to a POSH polypeptide. Generally, the POSH-AP may interact directly or indirectly with the POSH polypeptide. A preferred POSH-AP of the application is HERPUD1. Examples of HERPUD1 polypeptides are provided throughout.

As described, a POSH polypeptide interacts with the POSH-AP HERPUD1, a “homocysteine-inducible, endoplasmic reticulum stress-inducible, ubiquitin-like domain member 1” protein. This interaction was identified by Applicants as described herein below in a yeast two-hybrid assay. HERPUD1 is synonymous with Herp or HERP, and the terms are used interchangeably herein. HERPUD1 is involved in the maturation of an envelope virus, such as HIV.

Certain HERPUD1 polypeptides are involved in JNK-mediated apoptosis, particularly in vascular endothelial cells, including cells that are exposed to high levels of homocysteine. Certain HERPUD1 polypeptides are involved in the Unfolded Protein Response, a cellular response to the presence of unfolded proteins in the endoplasmic reticulum. Certain HERPUD1 polypeptides are involved in the regulation of sterol biosynthesis. Accordingly, certain POSH polypeptides are involved in the Unfolded Protein Response and sterol biosynthesis.

In other aspects, certain HERPUD1 polypeptides enhance presenilin-mediated amyloid beta-protein generation. For example, HERPUD1 polypeptides, when overexpressed in cells, increase the level of amyloid beta generation, and it has been observed that HERPUD1 polypeptides interact with the presenilin proteins, presenilin-1 (PS-1) and presenilin-2 (PS-2) (See Sai, X. et al (2002) J. Biol. Chem. 277:12915-12920). Accordingly, in certain aspects, POSH polypeptides may modulate the level of amyloid beta generation. Additionally, POSH polypeptides may interact with presenilin 1 and presenilin 2. Therefore, it is believed certain POSH polypeptides modulate presenilin-mediated amyloid beta generation. The accumulation of amyloid beta is one hallmark of Alzheimer's disease. Accordingly, these POSH polypeptides may be involved in the pathogenesis of Alzheimer's disease. At sites such as late intracellular compartment sites including the trans-Golgi network, certain mutant presenilin-2 polypeptides up-regulate production of amyloid beta peptides ending at position 42 (Aβ42). (See Iwata, H. et al (2001) J. Biol. Chem. 276: 21678-21685). Accordingly, POSH polypeptides may regulate production of Aβ42 through mutant presenilin-2 at late intracellular compartment sites including the trans-Golgi network. Furthermore, elevated homocysteine levels have been found to be a risk factor associated with Alzheimer's disease and cerebral vascular disease. Some risk factors, such as elevated plasma homocysteine levels, may accelerate or increase the severity of several central nervous system (CNS) disorders. Elevated levels of plasma homocysteine were found in young male patients with schizophrenia suggesting that elevated homocysteine levels could be related to the pathophysiology of aspects of schizophrenia (Levine, J. et al (2002) Am. J. Psychiatry 159:1790-2). Epidemiological and experimental studies have linked increased homocysteine levels with neurodegenerative conditions, including Alzheimer's disease, Parkinson's disease, depression, and stroke (reviewed in Mattson, M P and Shea, T B (2003) Trends Neurosci 26:137-46).

Accordingly, certain POSH polypeptides may be involved in neurological disorders. Neurological disorders include disorders associated with increased levels of plasma homocysteine, increased levels of amyloid beta production, or aberrant presenilin activity. Neurological disorders include CNS disorders, such as Alzheimer's disease, cerebral vascular disease, and schizophrenia.

Certain POSH polypeptides may be involved in cardiovascular diseases, such as thromboembolic vascular disease, and particularly the disease characteristics associated with hyperhomocysteinemia. See, for example, Kokame et al. 2000 J. Biol. Chem. 275:32846-53; Zhang et al. 2001 Biochem Biophys Res Commun 289:718-24.

As described herein, POSH and HERPUD1 are involved in viral maturation, including the production, post-translational processing, assembly and/or release of proteins in a viral particle. Accordingly, viral infections may be ameliorated by inhibiting an activity of HERPUD1 or POSH (e.g., inhibition of ubiquitin ligase activity). In preferred embodiments, the virus is a retroid virus, an RNA virus or an envelope virus, including HIV, Ebola, HBV, HCV, HTLV, West Nile Virus (WNV) or Moloney Murine Leukemia Virus (MMuLV). Additional viral species are described in greater detail below. In certain instances, a decrease of a POSH function is lethal to cells infected with a virus that employs POSH in release of viral particles.

In certain aspects, the application describes an hPOSH interaction with Rac, a small GTPase and the POSH associated kinases MLK, MKK and JNK. Rho, Rac and Cdc42 operate together to regulate organization of the actin cytoskeleton and the MLK-MKK-JNK MAP kinase pathway (referred to herein as the “JNK pathway” or “Rac-JNK pathway” (Xu et al., 2003, EMBO J. 2: 252-61). Ectopic expression of mouse POSH (“mPOSH”) activates the JNK pathway and causes nuclear localization of NF-κB. Overexpression of mPOSH in fibroblasts stimulates apoptosis. (Tapon et al. (1998) EMBO J. 17:1395-404). In Drosophila, POSH may interact with, or otherwise influence the signaling of, another GTPase, Ras. (Schnorr et al. (2001) Genetics 159: 609-22). The JNK pathway and NF-κB regulate a variety of key genes involved in, for example, immune responses, inflammation, cell proliferation and apoptosis. For example, NF-κB regulates the production of interleukin 1, interleukin 8, tumor necrosis factor and many cell adhesion molecules. NF-κB has both pro-apoptotic and anti-apoptotic roles in the cell (e.g., in FAS-induced cell death and TNF-alpha signaling, respectively). NF-κB is negatively regulated, in part, by the inhibitor proteins IκBα and IκBβ (collectively termed “IκB”). Phosphorylation of IκB permits activation and nuclear localization of NF-κB. Phosphorylation of IκB triggers its degradation by the ubiquitin system.

In an additional embodiment, a POSH polypeptide promotes nuclear localization of NF-κB. By downregulating POSH, apoptosis may be diminished in certain cells, and this will generally be desirable in conditions characterized by excessive cell death, such as myocardial infarction, stroke, degenerative diseases of muscle and nerve (particularly Alzheimer's disease), and for organ preservation prior to transplant.

In a further embodiment, a POSH polypeptide associates with a vesicular trafficking complex, such as a clathrin- or coatomer-containing complex, and particularly a trafficking complex that localizes to the nucleus and/or Golgi apparatus.

As described in WO03/095971A2 and WO03/078601A2, both herein incorporated by reference in their entirety, POSH polypeptides function as E3 enzymes in the ubiquitination system. Accordingly, downregulation or upregulation of POSH ubiquitin ligase activity can be used to manipulate biological processes that are affected by protein ubiquitination. Modulation of POSH ubiquitin ligase activity may be used to affect POSH and related biological processes, and likewise, modulation of POSH may be used to affect POSH ubiquitin ligase activity and related processes. Downregulation or upregulation may be achieved at any stage of POSH formation and regulation, including transcriptional, translational or post-translational regulation. For example, POSH transcript levels may be decreased by RNAi targeted at a POSH gene sequence. As another example, POSH ubiquitin ligase activity may be inhibited by contacting POSH with an antibody that binds to and interferes with a POSH RING domain or a domain of POSH that mediates interaction with a target protein (a protein that is ubiquitinated at least in part because of POSH activity).

As a further example, in a most preferred embodiment, small molecule inhibitors of POSH ubiquitin ligase activity are provided herein, consisting of compounds of the general formula I, more preferably, the Compounds 1 and 2.

As another example, POSH activity may be increased by causing increased expression of POSH or an active portion thereof. POSH, and POSH-APs that modulate POSH ubiquitin ligase activity may participate in biological processes including, for example, one or more of the various stages of a viral lifecycle, such as viral entry into a cell, production of viral proteins, assembly of viral proteins and release of viral particles from the cell. POSH may participate in diseases characterized by the accumulation of ubiquitinated proteins, such as dementias (e.g., Alzheimer's and Pick's), inclusion body myositis and myopathies, polyglucosan body myopathy, and certain forms of amyotrophic lateral sclerosis. POSH may participate in diseases characterized by excessive or inappropriate ubiquitination and/or protein degradation.

In certain aspects, the application provides methods and compositions for treatment of POSH-associated diseases (disorders), including neurological disorders. In certain aspects, the application provides methods and compositions for treatment of POSH-AP-associated diseases (disorders), such as HERPUD1-associated disorders, including neurological and viral disorders, as well as neurological disorders associated with unwanted apoptosis, including, for example a variety of neurodegenerative disorders, such as Alzheimer's disease.

Preferred therapeutics of the application for the treatment of a neurological disorder can function by disrupting the biological activity of a POSH polypeptide or POSH complex associated with a neurological disorder. Certain therapeutics of the application function by disrupting the activity of POSH by inhibiting the ubiquitin ligase activity of a POSH polypeptide, such as, for example, by inhibiting the POSH-mediated ubiquitination of HERPUD1.

In certain embodiments, the application relates to methods of treating or preventing neurological disorders. In certain aspects, the invention provides methods and compositions for the identification of compositions that interfere with the function of a POSH or a POSH-AP, such as HERPUD1, which function may relate to aberrant protein processing associated with a neurodegenerative disorder, such as for example, the processing of amyloid beta precursor protein associated with Alzheimer's disease.

Neurological disorders include disorders associated with increased levels of amyloid polypeptides, such as for example, Alzheimer's disease. Neurological disorders also include Parkinson's disease, Huntington's disease, schizophrenia, Pick's disease, Niemann-Pick's disease, prion-associated diseases (e.g., Mad Cow disease), depression, and schizophrenia.

In certain aspects, the present application provides assays for identifying therapeutic agents, which either interfere with or promote POSH or POSH-AP function. In certain aspects, the present application also provides assays for identifying therapeutic agents, which either interfere with or promote the complex formation between a POSH polypeptide and a POSH-AP polypeptide. In preferred embodiments of the application, the application provides assays for identifying therapeutic agents, which either interfere with or promote POSH or POSH-AP (e.g., HERPUD1) function. In certain preferred aspects, the present application also provides assays for identifying therapeutic agents, which either interfere with or promote the complex formation between a POSH polypeptide and a HERPUD1 polypeptide.

In preferred embodiments, the application provides agents for the treatment of neurological disorders: In certain embodiments, the application provides assays to identify, optimize or otherwise assess agents that disrupt the interaction between a POSH polypeptide and a HERPUD1 polypeptide.

In certain preferred embodiments, an agent of the application is one that disrupts a complex comprising POSH and HERPUD1. Optionally, the agent is one that disrupts a complex comprising POSH and HERPUD1 without inhibiting POSH ubiquitin ligase activity, such as POSH auto-ubiquitination. In certain embodiments, an agent of the application is one that inhibits POSH-mediated ubiquitination of HERPUD1, optionally without inhibiting POSH auto-ubiquitination.

In certain embodiments, agents of the application are useful in treating or preventing neurological disorders. Treatment or prevention of a neurological disorder includes inhibition of the progression of a neurological disorder. In certain embodiments, an agent useful in the treatment or prevention of a neurological disorder or an agent that inhibits the progression of a neurological disorder interferes with the ubiquitin ligase catalytic activity of POSH (e.g., POSH ubiquitination of a target protein such as HERPUD1).

In other embodiments, agents disclosed herein inhibit or promote POSH and POSH-AP, such as HERPUD1, mediated cellular processes such as protein processing in the secretory pathway, for example, processing of amyloid polypeptides.

In certain embodiments, agents of the application are antiviral agents, optionally interfering with viral maturation, and preferably where the virus is an envelope virus, and optionally a retroid virus or an RNA virus. In certain embodiments, an antiviral agent interferes with the interaction between POSH and a POSH-AP polypeptide, for example an antiviral agent may disrupt an interaction between a POSH polypeptide and a HERPUD1 polypeptide.

In yet additional embodiments, agents of the application interfere with the signaling of a GTPase, such as Rac or Ras, optionally disrupting the interaction between a POSH polypeptide and a Rac protein.

In certain embodiments, agents of the application interfere with the trafficking of a protein through the secretory pathway.

An additional POSH-AP may be added to a POSH ubiquitination assay to assess the effect of the POSH-AP (e.g., HERPUD1) on POSH-mediated ubiquitination and/or to assess whether the POSH-AP (e.g., HERPUD1) is a target for POSH-mediated ubiquitination.

The present application discloses reconstituted protein preparations including a POSH polypeptide and one or more interacting polypeptides.

Additional bioassays for assessing POSH and POSH-AP activities may include assays to detect the improper processing of a protein that is associated with a neurological disorder. One assay that may be used is an assay to detect the presence, including an increase or a decrease in the amount, of a protein associated with a neurological disorder. For example, the use of RNAi may be employed to knockdown the expression of a POSH or POSH-AP polypeptide, such as HERPUD1, in cells (e.g., CHO cells, COS cells, or HeLa cells). The production of a secreted protein such as for example, amyloid beta, in the cell culture media, can then be assessed and compared to production of the secreted protein from control cells, which may be cells in which the POSH or POSH-AP activity (e.g., HERPUD1 activity) has not been inhibited. In some instances, a label may be incorporated into a secreted protein and the presence of the labeled secreted protein detected in the cell culture media. Proteins secreted from any cell type may be assessed, including for example, neural cells.

Bioassays for POSH or POSH-AP activities may include assays to detect the improper processing of a protein that is associated with a degenerative neurological disorder, such as Alzheimer's disease. One assay that may be used to detect POSH or POSH-AP activity associated with a neurological disorder is an assay to detect the presence, including an increase or a decrease in the amount, of amyloid polypeptides. One such assay includes assessing the effect of modulation of a POSH or POSH-AP on the production of amyloid polypeptides. For example, the use of RNAi may be employed to knockdown the expression of a POSH polypeptide or a POSH-AP (e.g., HERPUD1) in cells (e.g., HeLa cells) that express proteins associated with gamma-secretase activity, such as presenilin (e.g., presenilin 1), which enzymatic activity is involved in the proteolytic cleavage of amyloid beta precursor protein (“APP”) to yield amyloid beta peptide. Optionally, other proteins associated with gamma-secretase may be expressed, such as, for example, nicastrin, Aph-1, and Pen-2. The production of amyloid polypeptides, e.g., in the cell culture media, can then be assessed and compared to the production of amyloid polypeptides from cells in which the POSH or POSH-AP activity has not been modulated. In certain embodiments, the levels of APP can be assessed and compared to the levels of APP in which POSH or POSH-AP activity has not been modulated.

Additional assays for POSH or POSH-AP activities include in vitro gamma-secretase assays, which may be employed to assess the effect of modulation of a POSH or POSH-AP (e.g., knockdown of POSH expression or knockdown of HERPUD1 expression by RNAi) on gamma-secretase activity in comparison to the gamma-secretase activity in cells in which the POSH or POSH-AP activity has not been modulated. For example, gamma-secretase activity in the cells in which POSH or POSH-AP activity has been modulated (e.g., by RNAi) may be monitored by incubating solubilized gamma-secretase from the cells with tagged (e.g., a FLAG epitope) APP-based substrate and detecting the substrates and cleavage products (e.g., amyloid beta peptide) by immunoblotting and comparing the results to those of control cells (cells in which the POSH or POSH-AP activity has not been modulated) manipulated in the same manner. The effect of modulation of an activity of a POSH polypeptide or a POSH-AP on amyloid polypeptide production may be assessed in any cell capable of producing amyloid polypeptides.

The effect of an agent that modulates the activity of POSH or a POSH-AP, such as HERPUD1, may be evaluated for effects on mouse models of various neurological disorders. For example, mouse models of Alzheimer's disease have been described. See, for example, U.S. Pat. No. 5,612,486 for “Transgenic Animals Harboring APP Allele Having Swedish Mutation”, U.S. Pat. No. 5,850,003 (the '003 patent) for “Transgenic Rodents Harboring APP Allele Having Swedish Mutation,”and U.S. Pat. No. 5,455,169 entitled “Nucleic Acids for Diagnosing and Modeling Alzheimer's Disease”. Mouse models of Alzheimer's disease tend to produce elevated levels of beta-amyloid protein in the brain, and the increase or decrease of such protein in response to treatment with a test agent may be detected. In some instances, it may also be desirable to assess the effects of a test, agent on cognitive or behavioral characteristics of a mouse model for Alzheimer's disease, as well as mouse models for other neurological disorders.

In a further embodiment, transcript levels may be measured in cells having higher or lower levels of POSH or POSH-AP activity, such as HERPUD1 activity, in order to identify genes that are regulated by POSH or POSH-APs. Promoter regions for such genes (or larger portions of such genes) may be operatively linked to a reporter gene and used in a reporter gene-based assay to detect agents that enhance or diminish POSH- or POSH-AP-regulated gene expression. Transcript levels may be determined in any way known in the art, such as, for example, Northern blotting, RT-PCR, microarray, etc. Increased POSH activity may be achieved, for example, by introducing a strong POSH expression vector. Decreased POSH activity may be achieved, for example, by RNAi, antisense, ribozyme, gene knockout, etc.

In certain embodiments, a test agent may be assessed for antiviral activity by assessing effects on an activity (function) of a POSH-AP, such as, for example, POSH. Activity (function) may be affected by an agent that acts at one or more of the transcriptional, translational or post-translational stages. For example, an siRNA directed to a POSH-AP encoding gene will decrease activity, as will a small molecule that interferes with a catalytic activity of a POSH-AP. In certain embodiments, the agent inhibits the activity of one or more POSH polypeptides.

The invention now being generally described, it will be more readily understood by reference to the following examples, which are included merely for purposes of illustration of certain aspects and embodiments of the present invention, and are not intended to limit the invention.

The invention now being generally described, it will be more readily understood by reference to the following examples, which are included merely for purposes of illustration of certain aspects and embodiments of the present invention, and are not intended to limit the invention.

### EXAMPLES

### I Biological Section

### Example 1

### Selection of POSH Inhibitors by HTS TR-FRET Assay

In order to test compounds as inhibitors of POSH, a ubiquitin protein ligase (E3) containing a RING domain that mediates its own ubiquitination in a RING finger-dependent manner in the presence of E1 and E2, a HTS (high-throughput screening) homogeneous TR-FRET (Time-Resolved Fluorescence Resonance Energy Transfer) assay was conducted to monitor POSH autoubiquitination.

The assay employs an ubiquitin-activating enzyme (E1) and an ubiquitin-conjugating enzyme (E2), a fused GST-RING subunit of POSH protein and two fluorophore-conjugated detection reagents, namely anti-GSTXL665 and europium cryptate-labeled ubiquitin. This homogeneous assay is based on FRET between a Eu³⁺ cryptate donor and a second fluorescent label (acceptor), allophycocyanin. Allophycocyanin, a 105 kDa phycobiliprotein, is crosslinked to ensure its stability. This chemically modified fluorophore, known as XL665, displays a set of photophysical properties matching those of Eu³⁺ cryptates.

The ubiquitination of POSH by ubiquitin cryptate and binding of the anti-GST tagged XL665 brings the fluorophores into close proximity allowing FRET reaction to occur (see diagram). The compounds that do not allow the FRET reaction to occur, are considered as inhibitors.

Self-ubiquitination of hPOSH was determined by homogenous time-resolved fluorescence resonance energy transfer assay (TR-FRET). The conjugation of ubiquitin cryptate to GST tagged hPOSH and the binding of anti-GST tagged XL665 bring the two fluorophores into close proximity, which allows the FRET reaction to occur. To measure hPOSH ubiquitination activity, GST tagged hPOSH (60 nM) was incubated in reaction buffer (40 mM Hepes-NaOH, pH 7.5, 1 mM DTT, 2 mM ATP, 5 mM MgCl2, (with recombinant E1 (8 nM), UbcH5c (500 nM), and ubiquitin-cryptate (15 nM) (CIS Bio International) for 30 minutes at 37° C. Reactions were stopped with 0.5M EDTA. Anti-GST-XL665 (CIS bio International) (50 nM) was then added to the reaction mixture for a further 45 minutes incubation at room temperature. Emission at 620 nm and 665 nm was obtained after excitation at 380 nm in a fluorescence reader (RUBYstar, BMG Labtechnologies). The generation of hPOSH-ubiquitin-cryptate adducts was then determined by calculating the fluorescence resonance energy transfer (FRET=(F)) using the following formula:

F=[(S665/S620−B665/B620)/(C665/C620−B665/B620)]

where: S=actual fluorescence, B=Fluorescence obtained in parallel incubation without cb1-b, C=Fluorescence obtained in reaction without added compounds.

In the first step, for evaluation and identification of POSH specific inhibitors, candidate compounds were added to the assay at various concentrations. The compounds that have blocked POSH autoubiquitination at a concentration of 10 μM (in DMSO solution), with inhibition rate of 90% or above, were designated as good inhibitor. The compounds (concentration of 1 μM) were again tested in an assay in the presence of both E1 and E2, but in the absence of the fused GST-RING subunit of POSH, and the compounds that inhibited E1+E2 ubiquitination above 70%, were removed. The compounds identified as good inhibitors of POSH autoubiquitination were subjected to optimization. Compound 1 presented an IC50 of 12 μM in this in vitro assay.

### Example 2

### Hit Optimization

All hits were classified by CSAR algorithms and manual SAR to design focused libraries. CSAR programs are readily available. Shih, for example provides “Shih” data miner (see http://www.shih.be).

This classification process is aimed at identifying structural determinants that affect potency and toxicity. The information gained in this step is employed for hit optimization. A focused library of 700-800 compounds (Asinex Ltd. Moscow, Russia; ChemDiv, San Diego USA) was then cherry-picked, collected and then screened for their effect on hPOSH-mediated autoubiquitination by employing the same assay as demonstrated in Example 1. The compound that was identified as good optimized hit is the compound herein identified as Compound 1 (Asinex Ltd.), whose chemical structure is depicted in Appendix A.

### Example 3

Assay for virus release—Compound 1 inhibits release of HIV-1 p24.

The POSH inhibitor Compound 1 was tested for its efficiency of viral budding and GAG expression and processing in treated and untreated Jurkat cells. The concentration of extracellular GAG p24 was used as an indication of viral budding.

Jurkat cells were incubated with Compound 1 for either 1 or 3 days. The next day, cells were transfected with the plasmid pNLenv1 (2 μg/ml). Virus-like particle (VLP) release was determined one day after transfection as follows: the culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The corresponding cells were washed three times with phosphate-buffered saline (PBS) and then solubilized by incubation on ice for 15 minutes in lysis buffer containing the following components: 50 mM Hepes-NaOH, (pH 7.5), 150 mM NaCl, 1.5 mM MgCl₂, 0.5% NP-40, 0.5% sodium deoxycholate, 1 mM EDTA, 1 mM EGTA and 1:200 dilution of protease inhibitor cocktail (EMD Biosciences, Inc.). The cell detergent extract was then centrifuged for 15 minutes at 14,000×g at 4° C. The VLP sample and a sample of the cleared cell extract, were resolved on a 12.5% SDS-polyacrylamide gel, then transferred onto nitrocellulose paper and subjected to immunoblot analysis with rabbit anti-CA antibodies (Seramun Diagnostica, GmbH). The CA was detected after incubation with a secondary anti-rabbit horseradish peroxidase-conjugated antibody, and detected by Enhanced Chemi-Luminescence (ECL) (Amersham Biosciences, Corp.) and detected by fluorescence imaging (Typhoon Instrument, Amersham Biosciences, Corp.). Compound 1 presented an IC50 of 100 μM in the virus release assay.

### Example 4

### POSH Protein-Protein Interactions by Yeast Two-Hybrid Assay

POSH-associated proteins were identified by using a yeast two-hybrid assay.

Procedure: Bait plasmid (GAL4-BD) was transformed into yeast strain AH109 (Clontech) and transformants were selected on defined media lacking tryptophan. Yeast strain Y187 containing pre-transformed Hela cDNA prey (GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.

Bait:

Plasmid vector: pGBK-T7 (Clontech)

Plasmid name: pPL269-pGBK-T7 GAL4 POSHdR

Protein sequence: Corresponds to aa 53-888 of POSH (RING domain deleted; SEQ ID NO: 1)

Library screened: Hela pretransformed library (Clontech).

The POSH-AP, HERPUD1 (Hs. 146393), was identified by yeast two-hybrid assay.

Examples of nucleic acid and amino acid sequences of HERPUD1 are provided below.

SEQ ID NO: 2—Human HERPUD1 cDNA sequence—var1 (public gi: 16507801)

SEQ ID NO: 3—Human HERPUD1 cDNA sequence—var2 (public gi: 10441910)

SEQ ID NO: 4—Human HERPUD1 cDNA sequence—var3 (public gi: 3005722)

SEQ ID NO: 5—Human HERPUD1 cDNA sequence—var4 (public gi: 21619176)

SEQ ID NO: 6—Human HERPUD1 cDNA sequence—var5 (public gi: 14249882)

SEQ ID NO: 7—Human HERPUD1 cDNA sequence—var6 (public gi: 12652674)

SEQ ID NO: 8—Human HERPUD1 cDNA sequence—var7 (public gi: 9711684)

SEQ ID NO: 9—Human HERPUD1 cDNA sequence—var8 (public gi: 3005718)

SEQ ID NO: 10—Human HERPUD1 cDNA sequence—var9 (public gi: 285960)

SEQ ID NO: 11—Human HERPUD1 cDNA sequence—var10 (public gi: 7661869)

SEQ ID NO: 12—Human HERPUD1 Protein sequence—var1 (public gi: 16507802)

SEQ ID NO: 13—Human HERPUD1 Protein sequence—var2 (public gi: 10441911)

SEQ ID NO: 14—Human HERPUD1 Protein sequence—var3 (public gi: 3005723)

SEQ ID NO: 15—Human HERPUD1 Protein sequence—var4 (public gi: 7661870)

SEQ ID NO: 16—Rat HERPUD1 cDNA sequence (public gi: 16758961)

SEQ ID NO: 17—Rat HERPUD1 Protein sequence (public gi: 16758962)

SEQ ID NO: 18—Mouse HERPUD1 cDNA sequence (public gi: 11612514)

SEQ ID NO: 19—Mouse HERPUD1 Protein sequence (public gi: 11612515)

### Example 5

HERPUD1 depletion by siRNA reduces HIV maturation.

HeLa SS6 cells were transfected with siRNA directed against HERPUD1 and with a plasmid encoding HIV proviral genome (pNLenv-1). Twenty-four hours post-HIV transfection, virus-like particles (VLP) secreted into the medium were isolated and reverse transcriptase activity was determined. HIV release of active RT is an indication for a release of processed and mature virus. When the levels of HERPUD1 were reduced, RT activity was inhibited by 80%, demonstrating the importance of HERPUD1 in HIV-maturation.

### Experimental Outline

Cell culture and transfection: HeLa SS6 were kindly provided by Dr. Thomas Tuschl (the laboratory of RNA Molecular Biology, Rockefeller University, New York, N.Y.). Cells were grown in Dulbecco's modified Eagle's medium (DMEM) supplemented with 10% heat-inactivated fetal calf serum and 100 U/ml penicillin and 100 μg/ml streptomycin. For transfections, HeLa SS6 cells were grown to 50% confluency in DMEM containing 10% FCS without antibiotics. Cells were then transfected With the relevant double-stranded siRNA (50-100 nM) (HERPUD1: 5′-GGGAAGUUCUUCGGAACCUdTdT-3′ (SEQ ID NO: 20) and 5′-dTdTCCCUUCAAGAAGCCUUGGA-5′ (SEQ ID NO: 21) using lipofectamin 2000 (Invitrogen, Paisley, UK). A day following the initial transfection cells were split 1:3 in complete medium and co-transfected 24 hours later with HIV-1NLenv1 (2 μg per 6-well) (Schubert et al., J. Virol. 72:2280-88 (1998)) and a second portion of double-stranded siRNA.

Assay for virus release: Virus and virus-like particle (VLP) release was determined one day after transfection with the proviral DNA as previously described (Adachi et al., J. Virol. 59: 284-91 (1986); Fukumori et al., Vpr. Microbes Infect. 2: 1011-17 (2000); Lenardo et al., J. Virol. 76: 5082-93 (2002)). The culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The resulting supernatant was removed and the viral-pellet was re-suspended in SDS-PAGE sample buffer. The corresponding cells were washed three times with phosphate-buffered saline (PBS) and then solubilized by incubation on ice for 15 minutes in lysis buffer containing the following components: 50 mM HEPES-NaOH, (pH 7.5), 150 mM NaCl, 1.5 mM MgCl₂, 0.5% NP-40, 0.5% sodium deoxycholate, 1 mM EDTA, 1 mM EGTA and 1:200 dilution of protease inhibitor cocktail (Calbiochem, La Jolla, Calif.). The cell detergent extract was then centrifuged for 15 minutes at 14,000×g at 4° C. The VLP sample and a sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected either after incubation with a secondary anti-rabbit horseradish peroxidase-conjugated antibody and detected by Enhanced Chemi-Luminescence (ECL) (Amersham Pharmacia) or after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry and the amount of released VLP was then determined by calculating the ratio between VLP-associated CA and intracellular CA and Pr55 as previously described (Schubert et al., J. Virol. 72:2280-88 (1998)).

Analysis of reverse transcriptase activity in supernatants: RT activity was determined in pelleted VLP (see above) by using an RT assay kit (Roche, Germany; Cat. No. 1468120). Briefly, VLP pellets were resuspended in 40 μl RT assay lysis buffer and incubated at room temperature for 30 minutes. At the end of incubation 20 μl RT assay reaction mix was added to each sample and incubation continued at 37° C. overnight. Samples (60 μl) were than transferred to MTP strip wells and incubated at 37° C. for 1 hour. Wells were washed five times with wash buffer and DIG-POD added for a one-hour incubation at 37° C. At the end of incubation wells were washed five times with wash buffer and ABST substrate solution was added and incubated until color developed. The absorbance was read in an ELISA reader at 405 nm (reference wavelength 492 nm). The resulting signal intensity is directly proportional to RT activity; RT concentration was determined by plotting against a known amount of RT enzyme included in separate wells of the reaction.

### Example 6

Amyloid precursor protein levels are reduced in cells that have reduced levels of POSH.

HeLa SS6 cells that express reduced levels of POSH (H153) and control cells expressing scrambled RNAi (H187) were transfected with a plasmid expressing amyloid precursor protein (APP) and presenilin 1 (PS1). Cells were metabolic labeled and protein extracts were immunoprecipitated with anti-amyloid beta specific antibody, which recognize an epitope common: to APP, C199 and Aβ polypeptides. A labeled protein was specifically precipitated by the antibody in H187-transfected cells (not shown). However, this polypeptide was not recognized in H153 cells (not shown) indicating that APP steady state levels are reduced in H153 and may be rapidly degraded in these cells.

### Methods

Cloning of pIRES-APP-PS1: Cloning was performed in two steps: Presenilin 1 (PS1) was first cloned from human brain library into pIREs (pIREs-PS1). Then APP-695 was obtained from amplifying two image clones (3639599 and 5582406) and mixing their PCR products in an additional PCR reaction to yield full-length APP695 that was further ligated into pIREs-PS1 to generate pIREs-APP-PS1.

### Transfection, Metabolic Labeling and Immunoisolation of Amyloid Beta (Aβ)

Hela SS6 cells expressing POSH-specific RNAi or scrambled RNAi (H153 and H187, respectively) were transfected with pIREs-APP-PS1 (24 μg) using lipofectamin 2000 reagent (Invitrogen, LTD). Twenty-four hours post-transfection, cells were metabolic labeled with 1 mCi of ³⁵S-methionine at 37° C. for an additional twenty-four hours. Media was collected from cells and spun at 3000 rpm for 10 min to pellet cell debris. Protease inhibitors and 2 mM 1,10-phenanthroline were added to the cleared cell media. Cells were lysed in lysis buffer (50 mM Tris-HCl, pH 7.8, 150 mM sodium chloride, 1 mM EDTA, 0.5% NP-40, 0.5% sodium deoxycholate and protease inhibitors). Cell media and lysate were immunoprecipitated with anti-Aβ (1-17) antibody (6E10) (Chemicon) or a non-relevant (NR) antibody. Precipitated proteins were separated on 16% Tris-Tricine gel. Gel was dried and bands detected by phosphoimager (Typhoon Instrument, Amersham Biosciences, Corp.).

### Example 7

### Cytoprotection Assay: Protection Conferred by Compounds 1 and 2 of Cells Infected by HIV-1 and HIV-2

For the HIV cytoprotection assay, CEM-SS cells were used and the viruses HIV-1IIIB, HIV-1RF, or HIV-2ROD.

Briefly, virus and cells were mixed in the presence of a test compounds and incubated for 6 days. The virus was pre-titered such that control wells exhibited 70 to 95% loss of cell viability due to virus replication. Therefore, antiviral effect or cytoprotection was observed when the compounds prevented virus replication. Each assay plate contained the following controls: cell control wells (cells only), virus control wells (cells plus virus), compound toxicity control wells (cells plus compound only), compound colorimetric control wells (compound only), as well as the experimental wells (compound plus cells plus virus). Cytoprotection and compound cytotoxicity were assessed by MTS (CellTiter® 96 Reagent, Promega, Madison Wis.) dye reduction, and the IC50 (concentration inhibiting virus replication by 50%), TC50 (concentration resulting in 50% cell death) and a calculated T1 (therapeutic index TC50/IC50) were obtained. Each assay included the HIV reverse transcriptase inhibitor AZT as a positive control.

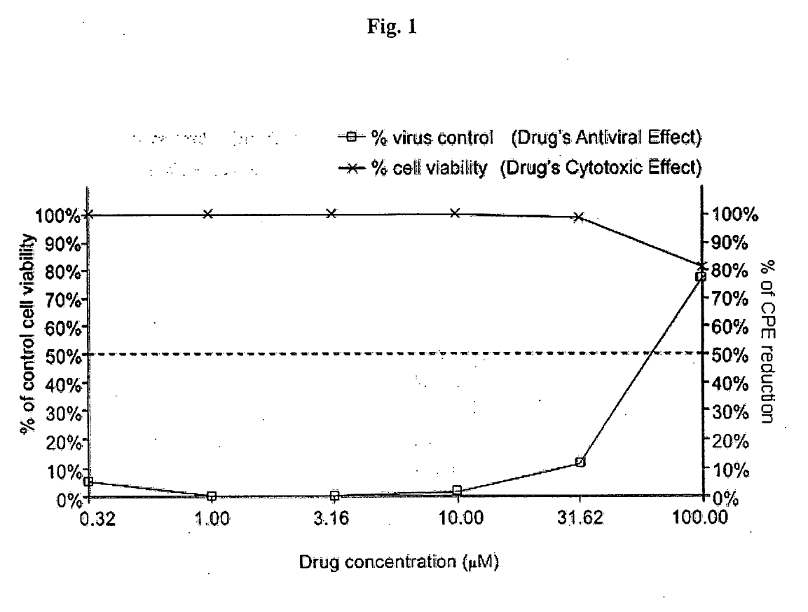

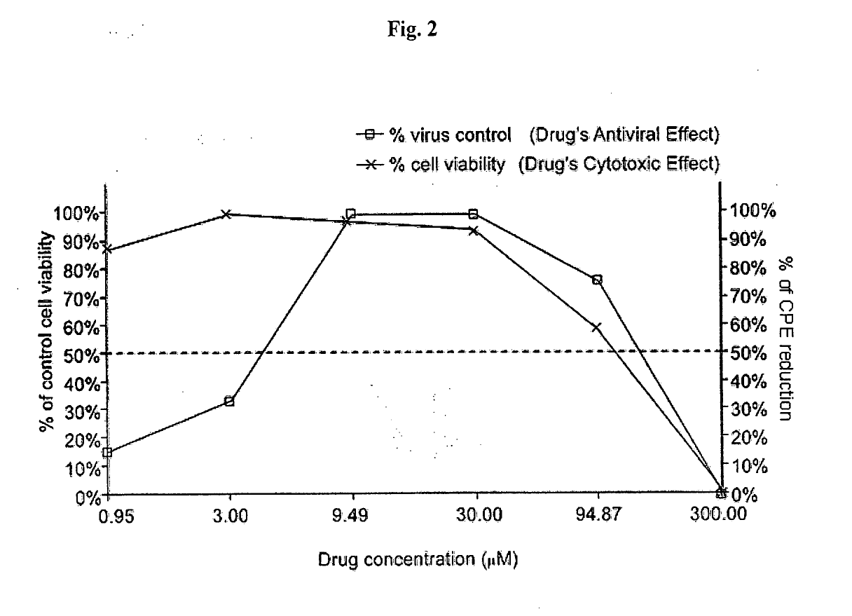

The IC50, TC50 and TI data obtained for cytoprotection by Compound 1 and Compound 2 against infection with HIV-1IIIB are depicted in Table 1, and the antiviral activity and compound cytotoxicity of Compound 1 and Compound 2 are shown in FIGS. 1 and 2, respectively. It is to be noted that Compound 2 was far more effective as anti-HIV-1IIIB agent compared to Compound 1.

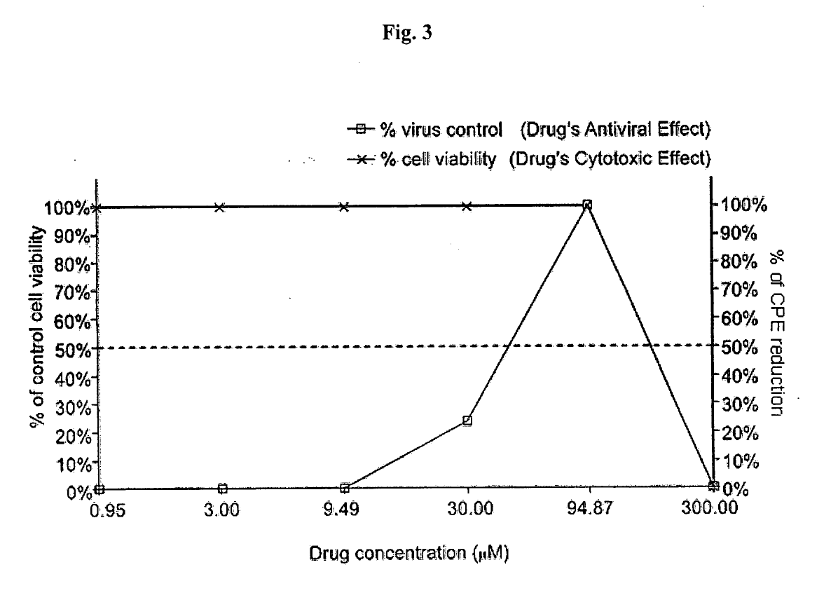

The cytoprotection assay data for Compound 2 against infection with HIV-2ROD are depicted in Table 2, and the antiviral activity and compound cytotoxicity are presented in FIG. 3.

TABLE 1
Protection by Compounds 1 and 2 against HIV-1IIIB in CEM-SS cells

| Compound | IC₅₀ | TC₅₀ | Antiviral Index (TI) |
|---|---|---|---|
| 1 | 62 μM | >100 μM | >1.61 |
| 2 | 3.99 μM | 114 μM | 28.5 |

TABLE 2
Protection of Compounds 2 against HIV-2ROD in CEM-SS cells

| IC₅₀ | TC₅₀ | Antiviral Index (TI) |
|---|---|---|
| 44.4 μM | 169 μM | 3.80 |

### II Chemical Section

### Example 8

### Synthesis of Compound 2

The synthesis of Compound 2 [2,5-dichloro-N-(4,6-dimethylpyridin-2-yl)benzenesulfonamide] was performed as follows (see Scheme 1 below):

To 2-amino-4,6-dimethylpyridine (300 mg, 2.5 mmol) dissolved in dry pyridine (2 ml), 2,5-dichlorobenzene-1-sulfonyl chloride (490 mg, 2 mmol) was added gradually while stirring. The reaction mixture was then heated on a steam bath for one hour. A solution of NaOH (100 mg, 2.5 mmol) in water (2 ml) was added slowly, and heating was continued for a short time. The pyridine was removed by distillation under reduced pressure, and water was added from time to time to maintain the volume in the reaction flask. Crude 2,5-dichloro-N-(4,6-dimethylpyridin-2-yl)benzenesulfonamide was separated as a yellow-brown solid, collected by filtration and recrystallized from AcOH to yield 410 mg (63%).

1H—NMR 11.87 (s, 1H), 8.20 (s, 1H), 7.26 (m, 1H), 7.37 (s, 2H), 7.28 (s, 1H), 6.74 (s, 1H), 6:37 (s, 1H), 2.46 (s, 3H), 2.28 (s, 3H) MS (EI): Calculated: 330/332/334. Found: [M]⁺=330/332/334.

Elemental Analysis: Calculated: Calculated: C, 47.14; H, 3.65; N, 8.46%. Found C, 46.99; H, 3.67; N, 8.46%. Light yellow solid, Melting Point: >174° C. (AcOH).

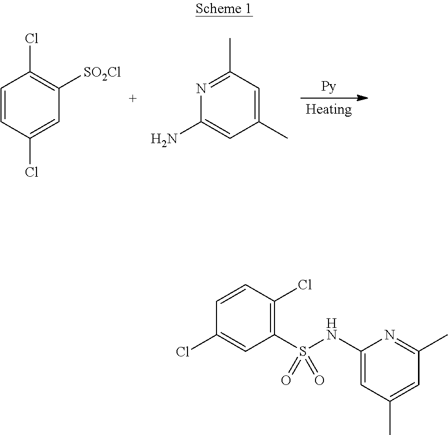

### Example 9

### Synthesis of Compound 1

The synthesis of Compound 1 [2,5-dichloro-N-(4,6-dimethylpyrimidin-2-yl)benzenesulfonamide] was performed in a similar manner to the synthesis of Compound 2, but using 2-amino-4,6-dimethylpyridine as the starting compound.

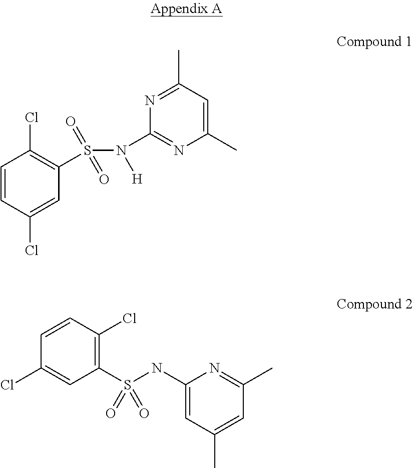

1. A pharmaceutical composition comprising a pharmaceutically acceptable carrier and a compound of the general formula I: 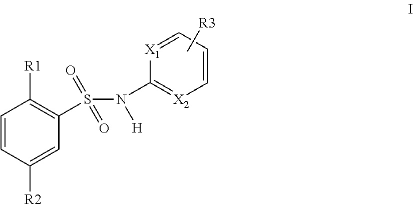
wherein
X₁ is N or C and X₂ is N;
R₁ and R₂ each independently is halogen, alkyl, aryl, heteroaryl, —COR₄, —COOR₄, —NR₅R₆, —CONR₅R₆ or —NR₇COR₈;
R₃ represents H or one to three radicals selected from lower alkyl, lower alkoxy, halogen, —COOR₄, —NR₅R₆ or —CONR₅R₆;
R₄ is H, hydrocarbyl or heterocyclyl;
R₅ and R₆ each independently is H, hydrocarbyl or heterocyclyl, or R₅ and R₆ together with the nitrogen atom to which they are attached form a saturated heterocyclic ring, optionally containing 1 or 2 further heteroatoms selected from N, S and O, and wherein said further N atom may be optionally substituted by lower alkyl unsubstituted or substituted by phenyl, halogen or hydroxy;
R₇ is H, lower alkyl or phenyl;
R₈ is aryl or heteroaryl;
wherein said lower alkyl, hydrocarbyl, heterocyclyl, aryl and heteroaryl is unsubstituted or substituted by one or more radicals selected from halogen, OR₄, —SR₄, —COR₄, —COOR₄, —NR₅R₆, —CONR₅R₆, NO₂, —NR₅—COR₄ —SO₃R₄, —SO₂R₄, —SO₂NR₅R₆ and —NR₅SO₂R₄, wherein R₄, R₅ and R₆ are as defined above;
wherein said hydrocarbyl is a straight or branched, acyclic or cyclic, saturated, unsaturated or aromatic, hydrocarbyl radical of 1-20 carbon atoms selected from an alkyl, alkenyl, alkynyl, carbocyclyl, aryl and an aralkyl radicals; said heteroaryl is a mono- or poly-cyclic heteroaromatic ring containing one to three heteroatoms selected from O, S and N; and said heterocyclyl is a saturated or partially unsaturated, unsubstituted or substituted, monocyclic, bicyclic or tricyclic heterocycle of 3-12 members in the ring(s), containing one to three heteroatom selected from O, S and N;
or an enantiomer or a pharmaceutically acceptable salt thereof, for the preparation of a medicament.

2. The pharmaceutical composition according to claim 1, wherein said halogen is F, Cl, Br or I; said hydrocarbyl is 1-10 carbon atoms; and said heterocyclyl is of 5-10 members in the ring(s).

3. The pharmaceutical composition according to claim 2, wherein said halogen is Cl; said hydrocarbyl is a C₁-C₄ lower alkyl selected from methyl, ethyl, n-propyl, isopropyl, n-butyl, sec-butyl, isobutyl and tert-butyl, optionally interrupted by one or more heteroatoms selected from O, S and/or N, or phenyl; said heteroaryl is selected from pyrrolyl, furyl, thienyl, pyrazolyl, imidazolyl, oxazolyl, thiazolyl, pyridyl, quinolinyl, isoquinolinyl, pyridazinyl, pyrimidinyl, pyrazinyl, 1,3,4-triazinyl, 1,2,3-triazinyl, 1,3,5-triazinyl, benzofuryl, isobenzofuryl, indolyl, imidazo[1,2-a]pyridyl, benzimidazolyl, benzthiazolyl, benzoxazolyl, and benzodiazepinyl; and said heterocyclyl is selected from dihydrofuryl, tetrahydrofuryl, dihydrothienyl, pyrrolydinyl, pyrrolynyl, dihydropyridyl, piperidinyl, piperazinyl, morpholino and 1,3-dioxanyl.

4. (canceled)

5. The pharmaceutical composition according to claim 3, wherein R₁ and R₂ are halogen and R₃ represents two lower alkyl radicals.

6. The pharmaceutical composition according to claim 5, wherein X₁ is C or N, X₂ is N, R₁ and R₂ are Cl and R₃ represents two methyl radicals.

7. The pharmaceutical composition according to claim 6, wherein the compound of general formula I is represented by Compound 1:

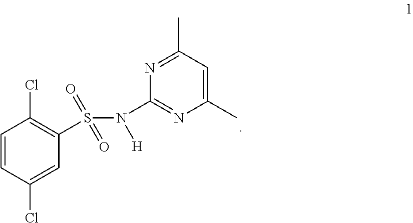

8. (canceled)

9. The pharmaceutical composition according to claim 6, wherein the compound of general formula I is represented by Compound 2:

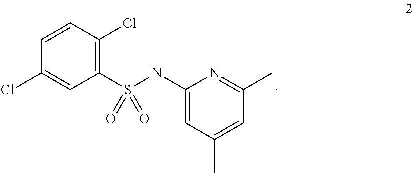

10. A method for inhibition of the ubiquitin ligase activity of a human polypeptide, which comprises administering to a subject in need a compound of formula I according to claim 1 in an amount effective for inhibiting the ubiquitin ligase activity of said human polypeptide.

11. The method according to claim 10, wherein the human polypeptide contains a RING domain.

12. The method according to claim 11, wherein the human polypeptide further comprises at least one SH3 domain.

13. The method according to claim 12, wherein said polypeptide is a human POSH polypeptide.

14. The method according to claim 13, for inhibiting the ubiquitination of a POSH-associated protein (POSH-AP).

15. The method according to claim 14, wherein said POSH-AP is HERPUD1.

16. The method according to claim 10, for treatment of a viral infection.

17. The method according to claim 16, wherein said viral infection is caused by a retroid virus selected from an RNA virus, an envelope virus, or a lentivirus, including primate lentivirus group.

18. The method according to claim 16, wherein said viral infection is caused by a virus selected from human immunodeficiency virus (HIV), human immunodeficiency virus type-1 (HIV-1), human immunodeficiency virus type-2 (HIV-2), hepatitis B virus (HBV), hepatitis C virus (HCV), Ebola virus, and human T-cell leukemia Virus (HTLV).

19. The method according to claim 18, for treatment of a viral infection caused by HIV-1 or HIV-2, comprising administering to a subject in need an effective amount of the compound of general formula I represented by Compound 1

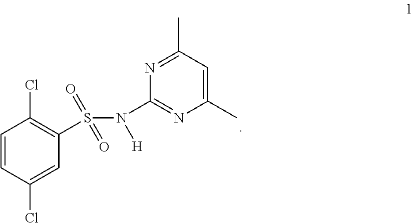

20. The method according to claim 18, for treatment of a viral infection caused by HIV-1 or HIV-2, comprising administering to a subject in need an effective amount of the compound of general formula 1 represented by Compound 2

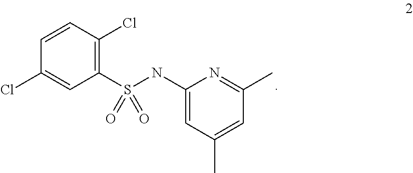

21. The method according to claim 10 for treatment of a neurological condition, disorder or disease.

22. The method according to claim 21, wherein said neurological disorder or disease is Alzheimer's disease, Parkinson's disease, Huntington's disease, Pick's disease, cerebral vascular disease, depression or schizophrenia.

23. The method according to claim 22, for treatment of Alzheimer's disease, comprising administering to a subject in need an effective amount of the compound of general formula I represented by Compound 1: 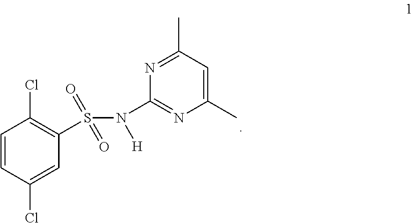
or Compound 2

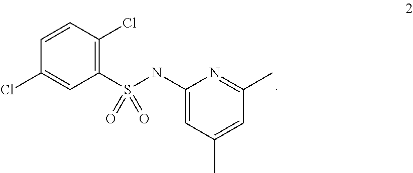

24. A compound of the formula: [chemical structure]
